(19) United States
(12) Patent Application Publication
(10) Pub. No.: US 2009/0130038 A1
(43) Pub. Date: May 21, 2009

(54) COSMETIC COMPOSITIONS COMPRISING AT LEAST ONE CATIONIC POLYMER AND AT LEAST ONE ESTER OF A C8-C24 FATTY ACID AND OXYETHYLENATED SORBITAN COMPRISING 2 TO 10 OXYETHYLENE MOTIFS, AND COSMETIC TREATMENT METHODS EMPLOYING SAID COMPOSITIONS

(76) Inventors: Olga Biganska, Asnieres (FR); Veronique Mahe, Vaux sur Seine (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/210,668

(22) Filed: Sep. 15, 2008

Related U.S. Application Data
(60) Provisional application No. 60/960,398, filed on Sep. 28, 2007.

(30) Foreign Application Priority Data
Sep. 14, 2007 (FR) 07/57593

(51) Int. Cl. A61K 8/84; A61Q 3/02

(52) U.S. Cl. 424/61

(57) ABSTRACT

The present disclosure relates to a composition comprising, in a cosmetically acceptable medium:
(i) at least one cationic polymer obtained by polymerizing a mixture of monomers comprising at least one vinylic monomer substituted with at least one amino group, at least one hydrophobic non-ionic vinylic monomer, and at least one associative vinylic monomer; and
(ii) at least one ester of a C₈-C₂₄ fatty acid and oxyethylenated sorbitan comprising 2 to 10 oxyethylene motifs.

These compositions may be used for treating keratinous materials.

This application claims benefit of U.S. Provisional Application No. 60/960,398, filed Sep. 28, 2007, the contents of which are incorporated herein by reference. This application also claims benefit of priority under 35 U.S.C. § 119 to French Patent Application No. FR 0757593, filed Sep. 14, 2007, the contents of which are also incorporated herein by reference.

Disclosed herein are novel cosmetic compositions for the treatment of keratinous materials, for example, human hair, comprising at least one cationic thickening polymer and at least one ester of a C₈-C₂₄ fatty acid and oxyethylenated sorbitan comprising 2 to 10 oxyethylene motifs, in a cosmetically acceptable medium. The present disclosure also relates to methods for cosmetic treatments of hair and scalp employing said cosmetic compositions.

To clean and/or wash keratinous materials such as human hair, detergent compositions (such as shampoos) comprising conventional anionic, non-ionic and/or amphoteric surfactants, but more particularly of the anionic type, are often used. Such compositions are applied to wet hair and the foam generated by massage or friction with the hands can, after rinsing with water, eliminate various types of dirt initially present on the hair or skin.

In addition to any cleaning properties and their cosmetic qualities, consumers want haircare compositions, in particular shampoos and conditioners, that are kind to the skin and the eyes, particularly in shampoos for children. In fact, the best shampoos may cause stinging in the eye when the dilute product flows into the eye socket, an event which frequently occurs with children. Further, people with sensitive skin experience discomfort such as redness, itching and stinging with many of such shampoos.

To overcome this set of problems described above, it is known to use esters of C₈-C₂₄ fatty acids and oxyethylenated sorbitan comprising 2 to 10 oxyethylene motifs as non-ionic surfactants in hair care compositions.

However, using cosmetic compositions comprising such oxyethylenated fatty acid esters had not been satisfactory, for example, regarding viscosity and/or distribution on the hair. The oxyethylenated fatty acid esters often render the cosmetic compositions too thick, thereby impeding their flow and/or their distribution on the hair because of the substantial chemical interactions which occur between said oxyethylenated fatty acid esters and other compounds such as conditioning agents present in said compositions.

Further, said compositions based on oxyethylenated fatty acid esters have an inhomogeneous texture and change substantially over time under normal storage conditions and as a function of temperature, especially their viscosity and their visual appearance. Thus, such compositions often have a cloudy appearance and a ropy texture at 45° C. under normal storage conditions.

Further, when such compositions based on oxyethylenated fatty acid esters also include anti-dandruff agents or agents producing a pearlized, iridescent, moiré or metallized appearance or effect such as cyclodextrin, such compositions are also not sufficiently stable.

Thus, there is a genuine need for developing cosmetic compositions containing esters of a C₈-C₂₄ fatty acid and oxyethylenated sorbitan comprising 2 to 10 oxyethylene motifs which do not suffer from the disadvantages described above, i.e. with good properties as regards use, a satisfactory visual appearance, and stable over time while providing satisfactory cosmetic properties when applied to keratinous materials, in particular the hair and scalp.

The Applicant has discovered that it is possible to formulate compositions for the cosmetic treatment of keratinous material having the desired properties by using in said compositions at least one ester of a C₈-C₂₄ fatty acid and oxyethylenated sorbitan comprising 2 to 10 oxyethylene motifs and at least one specific cationic thickening polymer, as defined below.

In fact, it has been shown that using said cationic polymer in the compositions of the present disclosure can improve properties as regards use, especially the viscosity, stability and texture of the cosmetic products such as shampoos and conditioners based on esters of C₈-C₂₄ fatty acids and oxyethylenated sorbitan comprising 2 to 10 oxyethylene motifs while providing the keratinous materials, for example, the hair, with satisfactory cosmetic properties, for example, as regards disentangling, manageability, softness, body and in particular sheen.

The compositions obtained have very good properties as regards use, i.e. the compositions can flow and be distributed more easily over the whole head.

Further, the stability on storage of the compositions of the present disclosure is also improved both at ambient temperature (20-25° C.) and at 45° C., in particular as regards their visual appearance and viscosity. The term “stable” as used in the present invention means that the visual appearance and the viscosity of said compositions does not change substantially over time under normal storage conditions, for example over 12 months, such as 24 months, and further such as 30 months, at ambient temperature following manufacture or, for example, for 2 months at 45° C. following manufacture.

Further, the compositions of the present disclosure have a non-ropy and soft texture which means that they can be more easily absorbed on the surface of the hair or skin. Such compositions also have a homogeneous texture.

Further, the improved texture means that, once deposited on the hair, it can remain there for a certain period without flowing off. This improved or even gelled texture means that smaller quantities of the products can be used.

Finally, when the compositions have a pearlizing agent such as cyclodextrin, the compositions are stable over time and retain their pearlized appearance.

Thus, the present disclosure pertains to compositions for the cosmetic treatment of keratinous materials, such as keratinous fibers, for example, human hair, comprising, in a cosmetically acceptable medium, at least one ester of a C₈-C₂₄ fatty acid and oxyethylenated sorbitan comprising 2 to 10 oxyethylene motifs and at least one cationic polymer as defined below.

The present disclosure also pertains to methods for cosmetic treatments of keratinous materials, such as keratinous fibers, for example, human hair, using the disclosed compositions.

The present disclosure also concerns the use of the disclosed compositions as shampoos or conditioners.

Other aims and characteristics, aspects and advantages of the present disclosure will become apparent from the following description and examples.

Unless otherwise indicated, the limits defining the extent of a range of values are included in this range of values. Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

The compositions for cosmetic treatments of keratinous materials, such as keratinous fibers, for example, human hair, comprise, in a cosmetically acceptable medium:

(i) at least one cationic polymer obtained by polymerizing a mixture of monomers comprising at least one vinylic monomer substituted with at least one amino group, at least one hydrophobic non-ionic vinylic monomer and at least one associative vinylic monomer;

(ii) at least one ester of a C₈-C₂₄ fatty acid and oxyethylenated sorbitan comprising 2 to 10 oxyethylene motifs.

The term “keratinous material” means the hair, eyelashes, eyebrows, skin, nails, mucosae or scalp and, for example, the hair, and the term “keratinous fibers” means the hair, eyelashes and eyebrows.

One of the characteristics of the present disclosure is the presence of at least one cationic polymer (i) which is obtained by polymerizing a mixture of monomers comprising at least one vinylic monomer substituted with at least one amino group, at least one hydrophobic non-ionic vinylic monomer and at least one associative vinylic monomer.

In at least one embodiment, the monomers comprising the cationic polymer (i) used in the present invention are different from each other.

According to one embodiment of the present disclosure, the at least one cationic polymer (i) is chosen from at least one thickening polymer. Within the context of the present disclosure, the term “thickening polymer” means a polymer which, when introduced in an amount of 1% into an aqueous or hydroalchoholic solution containing 30% ethanol and at a pH of 7, can attain a viscosity of at least 100 cps at 25° C. at a shear rate of 1 s⁻¹. This viscosity may be measured using a cone/plane viscosimeter (Haake R600 rheometer or the like).

According to one embodiment of the present disclosure, the presence of said at least one cationic polymer increases the viscosity of the compositions into which they are introduced by at least 50 cps at 25° C. and at a shear rate of 1 s⁻¹.

The at least one cationic polymer (i) used in the compositions of the present disclosure and their method of manufacture have been described in International Patent Application WO 2004/024779.

As used herein, and unless a different explanation is given:

The term “vinylic monomer” as used in the present disclosure means a monomer comprising at least one R₀CH═C(R₀)— group, in which each R₀ independently is chosen from a hydrogen atom, a C₁-C₃₀ alkyl group, —COOH, —CO—OR₀′, —O—CO—R₀′, —CO—NHR₀′, and —CO—NR₀′R₀″, wherein R₀′ and R₀″ are independently chosen from a C₁-C₃₀ alkyl group.

Thus, for example, within the context of the present disclosure, (meth)acrylates and (meth)acrylamides are vinylic monomers.

The vinylic monomers substituted with at least one amino group which can be used for the preparation of the at least one cationic polymer (i) used in the composition of the disclosure are monomers with an ethylenically unsaturated bond, which are basic and polymerizable. The at least one amino group may be derived from mono, di- and poly-amino alkyl groups or from heteroaromatic groups comprising a nitrogen atom. The at least one amino group may be chosen from a primary, secondary and tertiary amine. These monomers may be used in the form of an amine or in the form of a salt.

According to one embodiment of the present disclosure, the at least one vinylic monomer substituted with at least one amino group is chosen from:
mono(C₁-C₄ alkyl)amino(C₁-C₈ alkyl)(meth)acrylates;
di(C₁-C₄ alkyl)amino(C₁-C₈ alkyl)(meth)acrylates, such as di(C₁-C₄ alkyl)alkylamino(C₁-C₆ alkyl)(meth)acrylates;
mono(C₁-C₄ alkyl)amino(C₁-C₈ alkyl)(meth)acrylamides;
di(C₁-C₄ alkyl)amino(C₁-C₈ alkyl)(meth)acrylamides;
(meth)acrylamides comprising at least one heterocyclic group comprising a nitrogen atom;
(meth)acrylates comprising at least one heterocyclic group comprising a nitrogen atom;
nitrogen-containing heterocycles comprising at least one vinyl group;
and mixtures thereof.

Non-limiting examples of the at least one vinylic monomer substituted with at least one amino group include:
mono- or di-(C₁-C₄ alkyl)amino(C₁-C₄ alkyl)(meth)acrylates, such as 2-(N,N-dimethylamino)ethyl(meth)acrylate, 3-(N,N-dimethylamino)propyl(meth)acrylate, 4-(N,N-dimethylamino)butyl(meth)acrylate, (N,N-dimethylamino)-t-butyl(meth)acrylate, 2-(N,N-diethylamino)ethyl(meth)acrylate, 3-(N,N-diethylamino)propyl(meth)acrylate, 4-(N,N-diethylamino)butyl(meth)acrylate, 2-(N,N-dipropylamino)ethyl(meth)acrylate, 3-(N,N-dipropylamino)propyl(meth)acrylate or 4-(N,N-dipropylamino)butyl(meth)acrylate;
mono- or di(C₁-C₄ alkyl)amino(C₁-C₄ alkyl)(meth)acrylamides such as N′-(2-N,N-dimethylamino)ethyl(meth)acrylamide or N′-(3-N,N-dimethylamino)propyl acrylamide;
(meth)acrylamides or (meth)acrylates with a heterocyclic group containing a nitrogen atom, such as N-(2-pyridyl)acrylamide, N-(2-imidazolyl)methacrylamide, 2-(4-morpholinyl)ethyl methacrylate, 2-(4-morpholinyl)ethyl acrylate, N-(4-morpholinyl)methacrylamide or N-(4-morpholinyl)acrylamide; and
nitrogen-containing heterocycles comprising at least one vinyl group, such as 2-vinylpyridine or 4-vinylpyridine.

When the at least one monomer is in the form of a salt, it may be a mineral salt such as a hydrochloride, sulfate or phosphate; or a salt of an organic acid such as an acetate, maleate or fumarate salt.

In a particular embodiment, the at least one vinylic monomer substituted with at least one amino group is chosen from, by way of non-limiting example:
3-(N,N-dimethylamino)propyl(meth)acrylate;
N′-(3-N,N-dimethylamino)propyl(meth)acrylamide;
2-(N,N-dimethylamino)ethyl(meth)acrylate;
2-(N,N-diethylamino)ethyl(meth)acrylate;
2-(tert-butylamino)ethyl(meth)acrylate;
2-(N,N-dimethylamino)propyl(meth)acrylamide; and
2-(N,N-dimethylamino)neopentyl acrylate.

The at least one vinylic monomer substituted with at least one amino group may be present in an amount ranging from 10% to 70% by weight, such as 20% to 60% by weight, and further for example 30% to 40% by weight with respect to the total weight of the mixture of monomers.

The term “hydrophobic monomer” as used in the present disclosure means a monomer having a water solubility lower than 10 grams per 100 mL of water at 20° C.

The at least one hydrophobic non-ionic vinylic monomer which may be used for the preparation of the at least one cationic polymer (i) used in the composition of the disclosure are, in at least one embodiment, chosen from compounds of formula (I) or (II):

CH₂═C(X)Z, (I)

CH₂═CH—OC(O)R; (II)

wherein:
X is chosen from a hydrogen atom and a methyl group;
Z is chosen from the groups —C(O)OR¹, —C(O)NH₂, —C(O)NHR¹, —C(O)N(R¹)₂, —C₆H₅, —C₆H₄R¹, —C₆H₄OR¹, —C₆H₄Cl, —CN, —NHC(O)CH₃, —NHC(O)H, N-(2-pyrrolidonyl), N-caprolactamyl, —C(O)NHC(CH₃)₃, —C(O)NHCH₂CH₂—NH—CH₂CH₂-urea, —Si(R)₃, —C(O)O(CH₂)ₓSi(R)₃, —C(O)NH(CH₂)ₓSi(R)₃ and —(CH₂)ₓSi(R)₃;
x is an integer ranging from 1 to 6;
each R is independently chosen from a C₁-C₃₀ alkyl group;
each R¹ is independently chosen from a C₁-C₃₀ alkyl group, a hydroxylated C₂-C₃₀ alkyl group and a halogenated C₁-C₃₀ alkyl group.

Non-limiting examples of the at least one hydrophobic non-ionic vinylic monomer include C₁-C₃₀ alkyl(meth)acrylates, (C₁-C₃₀ alkyl)(meth)acrylamides; styrene, substituted styrenes, such as vinyltoluene (or 2-methylstyrene), butylstyrene, isopropylstyrene, para-chlorostyrene; vinyl esters such as vinyl acetate, vinyl butyrate, vinyl caprolate, vinyl pivalate and vinyl neodecanoate; unsaturated nitriles such as (meth)acrylonitrile and acrylonitrile; and unsaturated silanes, such as trimethylvinylsilane, dimethylethylvinylsilane, allyldimethylphenylsilane, allyltrimethylsilane, 3-acrylamidopropyltrimethylsilane, or 3-trimethylsilylpropyl methacrylate.

According to one embodiment of the present disclosure, the at least one hydrophobic non-ionic vinylic monomer is chosen from C₁-C₃₀ alkyl/acrylic acid esters, methacrylic acid/C₁-C₃₀ alkyl esters, and mixtures thereof, such as ethyl acrylate, methyl methacrylate, 3,3,5-trimethylcyclohexyl methacrylate and mixtures thereof.

The at least one hydrophobic non-ionic vinylic monomer may be present in an amount ranging from 20% to 80% by weight, such as 20% to 70% by weight and further for example 50% to 65% by weight with respect to the total weight of the mixture of monomers.

The at least one associative vinylic monomer which may be used to prepare the at least one cationic polymer (i) used in the composition of the present disclosure may be chosen, by way of non-limiting example, from compounds having an end (i′) with an ethylenically unsaturated bond or bonds for addition polymerization with other monomers of the system, a central polyoxyalkylene portion (ii′) to provide the polymers with their selective hydrophilic properties and a hydrophobic end (iii′) to provide the polymers with their selective hydrophobic properties.

The end (i′) with an ethylenically unsaturated bond or bonds of the associative vinylic monomer or monomers may be derived from a mono- or dicarboxylic acid anhydride with α,β-ethylenically unsaturated bond or bond, such as a C₃ or C₄ mono- or di-carboxylic acid or anhydride. Alternatively, the (i′) end of the at least one associative monomer may be derived from an allyl ether or from a vinyl ether; from a non-ionic urethane monomer substituted with a vinyl group such as that described U.S. Pat. No. Re. 33,156 or in U.S. Pat. No. 5,294,692; or a urea reaction product substituted with a vinyl group, such as that described in U.S. Pat. No. 5,011,978.

The central portion (ii′) of the at least one associative vinylic monomer or monomers is, for example a polyoxyalkylene segment comprising 5 to 250, 10 to 120, for example 15 to 60 C₂-C₇ alkylene oxide motifs. Central portions (ii′) include, by way of non-limiting example, polyoxyethylene, polyoxypropylene and polyoxybutylene segments comprising 5 to 150, such as 10 to 100, and further such as 15 to 60 ethylene oxide, propylene oxide or butylene oxide motifs and random or non-random sequences of ethylene oxide, propylene oxide or butylene oxide motifs. In one example, the central portions (ii)′ are polyoxyethylene segments.

The hydrophobic end (iii′) of the at least one associative vinylic monomer includes, for example, a hydrocarbon fragment chosen from a linear C₈-C₄₀ alkyl group, a C₂-C₄₀ alkyl group substituted with an aryl group, a phenyl group substituted with a C₂-C₄₀ alkyl group, a branched C₈-C₄₀ alkyl group, an alicyclic C₈-C₄₀ group and a C₈-C₈₀ complex ester.

The term “complex ester” as used in the present disclosure means an ester which differs from a simple ester.

The term “simple ester” as used in the present disclosure means any ester of a linear or branched, unsubstituted saturated C₁-C₃₀ aliphatic alcohol.

Non-limiting examples of hydrophobic ends (iii′) of the at least one associative vinylic monomer include linear and branched alkyl groups comprising 8 to 40 carbon atoms, such as capryl (C₈), isooctyl (branched C₈), decyl (C₁₀), lauryl (C₁₂), myristyl (C₁₄), cetyl (C₁₆), cetearyl (C₁₆-C₁₈), stearyl (C₁₈), isostearyl (branched C₁₈), arachidyl (C₂₀), behenyl (C₂₂), lignoceryl (C₂₄), cerotyl (C₂₆), montanyl (C₂₈), melissyl (C₃₀) and lacceryl (C₃₂).

Non-limiting examples of linear and branched alkyl groups comprising 8 to 40 carbon atoms and derivatives of a natural source are alkyl groups derived from hydrogenated ground nut oil, soya oil, canola oil (predominantly C₁₈), and hydrogenated C₁₆-C₁₈ tallow oil; and hydrogenated C₁₀-C₃₀ terpenols, such as hydrogenated geraniol (branched C₁₀), hydrogenated farnesol (branched C₁₅) and hydrogenated phytol (branched C₂₀).

Non-limiting examples of a phenyl group substituted with a C₂-C₄₀ alkyl group include the octylphenyl, nonylphenyl, decylphenyl, dodecylphenyl, hexadecylphenyl, octadecylphenyl, isooctylphenyl and sec-butylphenyl groups.

Non-limiting examples of C₈-C₄₀ alicyclic groups are groups derived from sterols of animal origin, such as cholesterol, lanosterol or 7-dehydrocholesterol; or groups derived from sterols of vegetable origin, such as phytosterol, stigmasterol or campesterol; or derivatives of sterols derived from microorganisms, such as ergosterol or mycosterol. Other examples of alicylic groups which may be used in the present disclosure include, for example, the cyclooctyl, cyclododecyl, adamantyl and decahydronaphtyl groups, and groups derived from natural alicylic compounds such as pinene, hydrogenated retinol, camphor and isobornyl alcohol.

Non-limiting examples of C₂-C₄₀ alkyl groups substituted with an aryl group include the 2-phenylethyl group, 2,4-diphenybutyl group, 2,4,6-triphenylhexyl group, 4-phenylbutyl group, 2-methyl-2-phenylethyl group and 2,4,6-tri(1′-phenylethyl)phenyl group.

Non-limiting examples of C₈-C₈₀ complex esters, such as C₈-C₄₀ complex esters, which may be used as the (iii′) end include hydrogenated castor oil (for example, the triglyceride of 12-hydroxystearic acid); 1,2-diacylglycerols such as 1,2-distearylglycerol, 1,2-dipalmitylglycerol, 1,2-dimyristylglycerol; di-, tri- or polyesters of sugars such as 3,4,6-tristearylglucose, 2,3-dilaurylfructose; and esters of sorbitan such as those described in U.S. Pat. No. 4,600,761.

The at least one associative vinylic monomer which may be used in the present disclosure may be prepared using any method which is known in the prior art, for example, the methods described in U.S. Pat. Nos. 4,421,902, 4,384,096, 4,514,552, 4,600,761, 4,616,074, 5,294,692, 5,292,843; 5,770,760 and 5,412,142.

According to one embodiment of the present disclosure, the at least one associative vinylic monomer or monomers which can be used in accordance with the disclosure are chosen from compounds with formula (III):

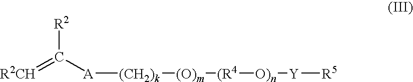

wherein:
each R² is independently chosen from a hydrogen atom, a methyl group, a —C(O)OH group, and a —C(O)OR³ group;
R³ is a C₁-C₃₀ alkyl group;
A is chosen from a —CH₂C(O)O—, —C(O)O—, —O—, —CH₂O—, —NHC(O)NH—, —C(O)NH—, —Ar—(CE₂)_{z}-NHC(O)O—, —Ar—(CE₂)_{z}-NHC(O)NH— and —CH₂CH₂—NHC(O)— group;
Ar is an arylene group;
E is chosen from a hydrogen atom and a methyl group;
z is an integer ranging from 0 to 1;
k is an integer ranging from 0 to 30;
m is an integer ranging 0 to 1, with the provisos that when k is 0, then m is 0, and
when k is an integer ranging from 1 to 30, then m is 1;
(R⁴—O)ₙ is a polyoxyalkylene group which is a homopolymer, a random copolymer or a block copolymer comprising C₂-C₄ oxyalkylene groups;
R⁴ is chosen from —C₂H₄—, —C₃H₆—, —C₄H₈— and mixtures thereof;
n is an integer ranging from 5 to 250;
Y is chosen from —R⁴O—, —R⁴NH—, —C(O)—, —C(O)NH—, R⁴NHC(O)NH—, and —C(O)NHC(O)—;
R⁵ is chosen from a substituted or unsubstituted alkyl group chosen from linear C₈-C₄₀ alkyl groups, branched C₈-C₄₀ alkyl groups, C₈-C₄₀ alicyclic groups, phenyl groups substituted with a C₂-C₄₀ alkyl group, C₂-C₄₀ alkyl groups substituted with an aryl group, and C₈-C₈₀ complex esters;
the alkyl group R⁵ optionally comprises at least one substituent chosen from hydroxy, alkoxy and halogeno groups.

According to one embodiment of the present disclosure, the at least one associative vinylic monomer is chosen from polyethoxylated cetyl(meth)acrylates, polyethoxylated cetearyl(meth)acrylates, polyethoxylated stearyl(meth)acrylates, polyethoxylated arachidyl(meth)acrylates, polyethoxylated behenyl(meth)acrylates, polyethoxylated lauryl(meth)acrylates, polyethoxylated cerotyl(meth)acrylates, polyethoxylated montanyl(meth)acrylates, polyethoxylated melissyl(meth)acrylates, polyethoxylated lacceryl(meth)acrylates, polyethoxylated 2,4,6-tri(1′-phenylethyl)phenyl(meth)acrylates, polyethoxylated hydrogenated castor oil(meth)acrylates, polyethoxylated canola(meth)acrylates, polyethoxylated cholesterol(meth)acrylates and mixtures thereof, wherein the polyethoxylated portion of the monomer comprises 5 to 100, such as 10 to 80 and further such as 15 to 60 ethylene oxide motifs.

According to a specific embodiment of the present disclosure, the at least one associative vinylic monomer is chosen from polyethoxylated cetyl methacrylates, polyethoxylated cetearyl methacrylates, polyethoxylated stearyl(meth)acrylates, polyethoxylated arachidyl(meth)acrylates, polyethoxylated behenyl(meth)acrylates, and polyethoxylated lauryl(meth)acrylates, wherein the polyethoxylated portion of the monomer comprises 10 to 80, such as 15 to 60 and further such as 20 to 40 ethylene oxide motifs.

According to certain embodiments of the present disclosure, the at least one associative vinylic monomer may be present in an amount ranging from 0.001% to 25% by weight, such as 0.01% to 15% by weight and further such as 0.1% to 10% by weight of the mixture of monomers.

The mixture of monomers for producing cationic polymers (i) may also contain at least one semi-hydrophobic monomer.

The at least one semi-hydrophobic vinylic surfactant monomer may moderate the associative properties of the cationic associative polymers which comprise them, thereby producing aqueous gels having a very good texture and very good rheological properties.

The term “semi-hydrophobic vinylic surfactant monomer” as used in the present disclosure means a structure similar to an associative monomer, but with a substantially non-hydrophobic end which thus does not provide the polymers with an associative property.

The associative property of a polymer is linked to the property in a given medium of the molecules of said polymer to associate together or to associate with molecules of a co-agent (such as a surfactant) which results, in a certain concentration range, in a supplemental increase in the viscosity of the medium.

The at least one semi-hydrophobic vinylic surfactant monomer is generally a compound comprising two portions:
(i″) an unsaturated terminal group to allow addition polymerization with other monomers of the reaction mixture; and
(ii″) a polyoxyalkylene group to attenuate associations between the hydrophobic groups of the polymer or the hydrophobic groups of other materials which may be present in the composition containing the polymer.

The end providing the vinylically or ethylenically unsaturated bond for addition polymerization may be derived, for example, from a mono- or di-carboxylic acid or anhydride with an α,β-ethylenically unsaturated bond, such as a C₃-C₄ mono- or di-carboxylic acid or an anhydride of said acid. In another embodiment, the (i″) end may be derived from an allyl ether, a vinyl ether or from a non-ionic unsaturated urethane.

The polymerizable unsaturated end (i″) may also be derived from a C₈-C₃₀ unsaturated fatty acid comprising at least one free carboxy functional group. This C₈-C₃₀ group forms part of the unsaturated (i″) end and is different from the pendant hydrophobic groups of the at least one associative monomer, which are separated from the unsaturated end of the associative monomer by a hydrophilic spacer group.

The polyoxyalkylene portion (ii″) comprises a long chain polyoxyalkylene segment which is similar to the hydrophilic portion of the at least one associative monomer. Non-limiting examples of polyoxyalkylene portions (ii″) include C₂-C₄ polyoxyethylene, polyoxypropylene and polyoxybutylene motifs comprising 5 to 250, such as 10 to 100 oxyalkylene motifs. When the at least one semi-hydrophobic vinylic surfactant monomer comprises more than one type of oxyalkylene motif, these motifs may be disposed in a random, non random or block sequence.

In at least one embodiment, the at least one semi-hydrophobic vinylic surfactant monomer is chosen from compounds of formulae (IV) and (V):

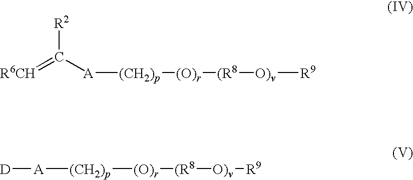

wherein:
R⁶ is independently chosen from a hydrogen atom, a C₁-C₃₀ alkyl group, —C(O)OH, or —C(O)OR⁷;
R⁷ is a C₁-C₃₀ alkyl group;
A is chosen from —CH₂C(O)O—, —C(O)O—, —O—, —CH₂O—, —NHC(O)NH—, —C(O)NH—, —Ar—(CE₂)_{z}-NHC(O)O—, —Ar—(CE₂)_{z}-NHC(O)NH— and —CH₂CH₂NHC(O)—;
Ar is an arylene group;
E is chosen from a hydrogen atom and a methyl group;
z is an integer ranging from 0 to 1;
p is an integer ranging from 0 to 30;
r is an integer ranging from 0 to 1, with the provisos that when p is 0, then r is 0, and when p is an integer ranging from 1 to 30, then r is 1
(R₈—O)ᵥ is a polyoxyalkylene which is a homopolymer, a random copolymer or a block copolymer comprising C₂-C₄ oxyalkylene motifs, where R⁸ is chosen from —C₂H₄—, —C₃H₆—, —C₄H₈— and mixtures thereof, and v is an integer ranging from 5 to 250;
R⁹ is chosen from a hydrogen atom and a C₁-C₄ alkyl;
D is chosen from a C₈-C₃₀ alkenyl group optionally substituted with a carboxy group.

In at least one embodiment of the present disclosure, the mixture of monomers may comprise at least one semi-hydrophobic vinylic surfactant monomer chosen from one of the following formulae:

CH₂═CH—O(CH₂)ₐO(C₃H₆O)_{b}(C₂H₄O)_{c}H; and

CH₂═CHCH₂O(C₃H₆O)_{d}(C₂H₄O)ₑH;

wherein:
a is an integer ranging from 2 to 4;
b is an integer ranging from 1 to 10;
c is an integer ranging from 5 to 50;
d is an integer ranging from 1 to 10; and
e is an integer ranging from 5 to 50.

Non-limiting examples of the at least one semi-hydrophobic vinylic surfactant monomer includes, for example, polymerizable emulsifying agents sold under reference numbers EMULSOGEN® R109, R208, R307, RAL109, RAL208 and RAL307 by CLARIANT; BX-AA-E5P5 sold by BIMAX; and MAXEMUL® 5010 and 5011 sold by UNIQEMA.

According to the Manufacturers:

EMULSOGEN® R109 is a randomly ethoxylated/propoxylated 1,4-butanediol vinyl ether with empirical formula:

CH₂═CH—O(CH₂)₄O(C₃H₆O)₄(C₂H₄O)₁₀H;

EMULSOGEN® R208 is a randomly ethoxylated/propoxylated 1,4-butanediol vinyl ether with empirical formula:

CH₂═CH—O(CH₂)₄O(C₃H₆O)₄(C₂H₄O)₂₀H;

EMULSOGEN® R307 is a randomly ethoxylated/propoxylated 1,4-butanediol vinyl ether with empirical formula:

CH₂═CH—O(CH₂)₄O(C₃H₆O)₄(C₂H₄O)₃₀H;

EMULSOGEN® RAL 109 is a randomly ethoxylated/propoxylated allyl ether with empirical formula:

CH₂═CHCH₂—O(C₃H₆O)₄(C₂H₄O)₁₀H;

EMULSOGEN® RAL 208 is a randomly ethoxylated/propoxylated allyl ether with empirical formula:

CH₂═CHCH₂—O(C₃H₆O)₄(C₂H₄O)₂₀H;

EMULSOGEN® RAL 307 is a randomly ethoxylated/propoxylated allyl ether with empirical formula:

CH₂═CHCH₂—O(C₃H₆O)₄(C₂H₄O)₃₀H;

MAXEMUL® 5010 is a hydrophobic carboxylated C₁₂-C₁₅ alkenyl ethoxylated with 24 ethylene oxide units;

MAXEMUL® 5011 is a hydrophobic carboxylated C₁₂-C₁₅ alkenyl ethoxylated with 34 ethylene oxide units;

and BX-AA-E5P5 is a randomly ethoxylated/propoxylated allyl ether with empirical formula:

CH₂═CHCH₂—O(C₃H₆O)₅(C₂H₄O)₅H.

The amount of the at least one semi-hydrophobic vinylic surfactant monomer used in preparing the at least one cationic polymer, such as the at least one thickening agent, may vary widely and depends on the final rheological properties desired for the polymer. The at least one semi-hydrophobic vinylic surfactant may be present in an amount ranging from 0 to 25% by weight, such as 0.01% to 25% by weight, and further such as 0.1% to 10% by weight, with respect to the total weight of the monomer mixture.

The at least one cationic polymer (i) used in the composition of the disclosure are prepared from a mixture of monomers which may contain at least one hydroxylated non-ionic vinylic monomer.

These monomers are monomers with an ethylenically unsaturated bond comprising at least one hydroxy substituent.

Non-limiting examples of hydroxylated non-ionic vinylic monomers include C₁-C₆ hydroxyalkyl(meth)acrylates, such as C₁-C₄ hydroxyalkyl(meth)acrylates, such as 2-hydroxyethyl methacrylate (HEMA), 2-hydroxyethyl acrylate (2-HEA) or 3-hydroxypropyl acrylate; (C₁-C₄ hydroxyalkyl)(meth)acrylamides such as N-(2-hydroxyethyl)methacrylamide, N-(2-hydroxyethyl)acrylamide, N-(3-hydroxypropyl)acrylamide or N-(2,3-dihydroxypropyl)acrylamide; and mixtures thereof. Allyl alcohol, the monoallyl ether of glycerol, 3-methyl-3-buten-1-ol, vinyl alcohol precursors and their equivalents such as vinyl acetate, may also be mentioned.

The at least one hydroxylated non-ionic vinylic monomer may be present in an amount ranging from 0% to 10% by weight of the total weight of the mixture of monomers. In one embodiment, the at least one Thydroxylated non-ionic vinylic monomer is present in an amount ranging from 0.01% to 10% by weight, such as 1% to 8% by weight, and further such as 1% to 5% by weight relative to the total weight of the mixture of monomers.

The at least one cationic polymer (i) used in the composition of the disclosure is prepared from a mixture of monomers which may comprise at least one cross-linking monomer allowing the introduction of branches and allowing the molecular mass to be controlled.

Non-limiting examples of polyunsaturated cross-linking agents which may be used herein include mono-unsaturated compounds with a reactive group capable of cross-linking a copolymer which has already been formed during or after polymerization may also be used; and poly functional monomers containing multiple reactive groups such as epoxy groups, isocyanates and hydrolysable silane groups. Many polyunsaturated compounds may be employed to generate a partially or substantially cross-linked three dimensional network.

Non-limiting examples of polyunsaturated cross-linking monomers which may be used include polyunsaturated aromatic monomers such as divinylbenzene, divinyinaphtylene or trivinylbenzene; polyunsaturated alicyclic monomers such as 1,2,4-trivinylcyclohexane; bi functional esters of phthalic acid such as diallyl phthalate; polyunsaturated aliphatic monomers such as dienes, trienes or tetraenes, such as isoprene, butadiene, 1,5-hexadiene, 1,5,9-decatriene, 1,9-decadiene or 1,5 heptadiene.

Other non-limiting examples of cross-linking polyunsaturated monomers which may be used include polyalkenyl ethers such as triallylpentaerythritol, diallylpentaerythritol, diallylsaccharose, octaallylsaccharose or the diallyl ether of trimethylolpropane; polyunsaturated esters of poly alcohols or poly acids such as 1,6-hexanediol di(meth)acrylate, tetramethylene tri(meth)acrylate, allyl acrylate, diallyl itaconate, diallyl fumarate, diallyl maleate, trimethylolpropane tri(meth)acrylate, trimethylolpropane di(meth)acrylate or polyethylene glycol di(meth)acrylate; alkylene-bisacrylamides such as methylene-bisacrylamide or propylene-bisacrylamide; hydroxylated and carboxylated derivatives of methylene-bisacrylamide, such as N,N′-bismethylol-methylene-bisacrylamide; polyethyleneglycol di(meth)acrylates, such as ethyleneglycol di(meth)acrylate, diethyleneglycol di(meth)acrylate or triethyleneglycol di(meth)acrylate, polyunsaturated silanes such as dimethyldivinylsilane, methyltrivinylsilane, allyldimethylvinylsilane, diallyidimethylsilane or tetravinylsilane; and polyunsaturated stannanes such as tetraallyltin or diallyidimethyltin.

Non-limiting example of mono-unsaturated cross-linking monomers which carry a reactive group that may be used according to the present disclosure include N-methylolacrylamides; N-alcoxy(meth)acrylamides, wherein the alkoxy group comprises 1 to 18 carbon atoms; and unsaturated hydrolysable silanes such as triethoxyvinylsilane, tris-isopropoxyvinylsilane and 3-triethoxysilylpropyl methacrylate.

Non-limiting examples of polyfunctional cross-linking monomers which contain a plurality of reactive groups include hydrolysable silanes such as ethyltriethoxysilane or ethyltrimethoxysilane; hydrolysable epoxy silanes such as 2-(3,4-epoxycyclohexyl)ethyltriethoxysilane or 3-glycidoxypropyltrimethoxysi lane; polyisocyanates such as 1,4-diisocyanatobutane, 1,6-diisocyanatohexane, 1,4-phenylenediisocyanate or 4,4′-oxybis(phenyl)socyanate); unsaturated epoxides such as glycidyl methacrylate or allylglycidylether; and polyepoxides such as diglycidylether, 1,2,5,6-diepoxyhexane, or ethyleneglycol diglycidylether.

In at least one embodiment, polyunsaturated cross-linking monomers that may be used include, for example, ethoxylated polyols such as diols, triols and bis-phenols, ethoxylated with 2 to 100 moles of ethylene oxide per mole of hydroxylated functional group and terminated by a polymerizable unsaturated group such as a vinylether, an allylether, an acrylate ester or a methacrylate ester. Examples of such cross-linking monomers are ethoxylated bisphenol A dimethacrylate, ethoxylated bisphenol F dimethacrylate and ethoxylated trimethylolpropane trimethacrylate.

Other non-limiting examples of include the cross-linking agents derived from ethoxylated polyols described in U.S. Pat. No. 6,140,435.

In at least one embodiment, examples of cross-linking monomers include the acrylate and methacrylate esters of polyols comprising at least two acrylate or methacrylate ester groups, such as trimethylolpropane triacrylate (TMPTA), trimethylolpropane dimethacrylate, triethylene glycol dimethacrylate (TEGDMA), or ethoxylated (30) bisphenol A dimethacrylate (EOBDMA).

The at least one cross-linking monomer may be present in an amount ranging from 0% to 5% by weight with respect to the mixture of monomers. In at least one embodiment, the at least one cross-linking monomer is present in an amount ranging from 0.001% to 5% by weight, such as 0.05% to 2% by weight, and further such as 0.1% to 1% by weight relative to the total weight of the mixture of monomers.

The mixture of monomers may also comprise at least one chain transfer agent. Non-limiting examples of the at least one chain transfer agentinclude thiol compounds, disulfide compounds such as C₁-C₁₈ mercaptans, mercaptocarboxylic acids, mercaptocarboxylic acid esters, thioesters, (C₁-C₁₈ alkyl)disulfides, aryldisulfides, polyfunctional thiols; phospites and hypophosphites; halogenoalkane compounds such as carbon tetrachloride, borotrichloromethane; and unsaturated chain transfer agents such as alpha-methylstyrene.

Non-limiting examples of polyfunctional thiols are trifunctional thiols such as trimethylolpropane-tris-(3-mercaptopropionate), tetrafunctional thiols such as pentaerythritol-tetra-(3-mercaptopropionate), pentaerythritol-tetra-(thioglycolate) or pentaerythritol-tetra(thiolactate); and hexafunctional thiols, such as pentaerythritol-hexa-(thioglyconate).

Other non-limiting examples of the at least one chain transfer agent include catalytic chain transfer agents which reduce the molecular weight of the addition polymers during free radical polymerization of the vinylic monomers, such as cobalt complexes, for example cobalt (II) chelates. The at least one chain transfer agent may frequently be used at concentrations which are low compared with thiol-containing chain transfer agents.

Non-limiting examples of the at least one chain transfer agent includes octyl mercaptan, n-dodecyl mercaptan, t-dodecyl mercaptan, hexadecyl mercaptan, octadecyl mercaptan (ODM), isooctyl 3-mercaptopropionate (IMP), butyl 3-mercaptopropionate, 3-mercaptopropionic acid, butyl thioglycolate, isooctyl thioglycolate and dodecyl thioglycolate.

The chain transfer agent or agents may be added to the mixture of monomers in an amount ranging from 0% to 10% relative to the total weight of the mixture of monomers. In at least one embodiment, the at least one chain transfer agent is present in an amount ranging from 0.1% to 5% by weight relative to the total monomer weight.

The mixture of monomers allowing the preparation of the at least one cationic polymer (i) used in the composition of the disclosure may comprise at least one polymeric stabilizing agent in order to obtain stable dispersions or emulsions. The at least one stabilizing polymer may be soluble in water. Non-limiting examples include synthetic polymers, such as polyvinyl alcohols, partially hydrolyzed polyvinyl acetates, polyvinylpyrrolidone, polyacrylamides, polymethacrylamides, carboxylated addition polymers, polyalkyl vinyl ethers; natural hydrosoluble polymers, such as gelatin, peptins, alginates, casein or starch; and modified natural polymers such methylcellulose, hydroxypropylcellulose, carboxymethylcellulose or allyl hydroxyethylcelluloses.

The at least one polymeric stabilizing agent may be present in an amount ranging from 0% to 2% by weight relative to the total weight of the mixture of monomers, such as 0.0001% to 1% by weight, and further such as 0.01% to 0.5% by weight relative to the total weight of the mixture of monomers.

In another embodiment, the mixture of monomers comprises, with respect to the total weight of the mixture of monomers:
a) from 10% to 70% by weight of at least one vinylic monomer substituted with at least one amino group;
b) from 20% to 80% by weight of at least one hydrophobic non-ionic vinylic monomer;
c) from 0.001% to 25% by weight of at least one associative vinylic monomer;
d) from 0% to 25% by weight of at least one semi-hydrophobic vinylic surfactant monomer;
e) from 0% to 10% by weight of at least one hydroxylated non-ionic vinylic monomer;
f) from 0% to 5% by weight of at least one cross-linking monomer;
g) from 0% to 10% by weight of at least one chain transfer agent; and
h) from 0% to 2% by weight of at least one polymeric stabilizing agent.

In another embodiment, the mixture of monomers comprises, with respect to the total weight of the mixture of monomers:
a) from 20% to 60% by weight of at least one vinylic monomer substituted with at least one amino group;
b) from 20% to 70% by weight of at least one hydrophobic non-ionic vinylic monomer;
c) from 0.01% to 15% by weight of at least one associative vinylic monomer;
d) from 0.1% to 10% by weight of at least one semi-hydrophobic vinylic surfactant monomer;
e) from 0.01% to 10% by weight of at least one hydroxylated non-ionic vinylic monomer;
f) from 0.001% to 5% by weight of at least one cross-linking monomer;
g) from 0.001% to 10% by weight of at least one chain transfer agent; and
h) from 0% to 2% by weight of at least one polymeric stabilizing agent.

In another embodiment, the mixture of monomers allowing the preparation of the at least one cationic polymer (i) used in the composition of the disclosure comprises, with respect to the total weight of the mixture of monomers:

a) from 20% to 50% by weight of at least one vinylic monomer substituted with at least one amino group, chosen from:
3-(N,N-dimethylamino)propyl(meth)acrylate;
N′-(3-N,N-dimethylamino)propyl(meth)acrylamide;
2-(N,N-dimethylamino)ethyl(meth)acrylate;
2-(N,N-diethylamino)ethyl(meth)acrylate;
2-(tert-butylamino)ethyl(meth)acrylate;
2-(N,N-dimethylamino)propyl(meth)acrylamide; and
2-(N,N-dimethylamino)neopentyl acrylate;

b) from 50% to 65% by weight of at least one hydrophobic non-ionic vinylic monomer chosen from acrylic acid/C₁-C₃₀ alkyl esters, methacrylic acid/C₁-C₃₀ alkyl esters, and mixtures thereof;

c) from 0.1% to 10% by weight of at least one associative vinylic monomer chosen from polyethoxylated cetyl methacrylates, polyethoxylated cetearyl methacrylates, polyethoxylated stearyl(meth)acrylates, polyethoxylated arachidyl(meth)acrylates, polyethoxylated behenyl(meth)acrylates, polyethoxylated lauryl(meth)acrylates, polyethoxylated cerotyl(meth)acrylates, polyethoxylated montanyl(meth)acrylates, polyethoxylated melissyl(meth)acrylates, polyethoxylated lacceryl(meth)acrylates, polyethoxylated 2,4,6-tri(1′-phenylethyl)phenyl(meth)acrylates, polyethoxylated hydrogenated castor oil(meth)acrylates, polyethoxylated canola(meth)acrylates, polyethoxylated cholesterol(meth)acrylates and mixtures thereof

d) from 0.1% to 10% by weight of at least one semi-hydrophobic vinylic surfactant monomer chosen from one of the following formulae:

CH₂═CH—O(CH₂)ₐO(C₃H₆O)_{b}(C₂H₄O)_{c}H, and

CH₂═CHCH₂O(C₃H₆O)_{d}(C₂H₄O)ₑH

wherein:
a is an integer ranging from 2 to 4;
b is an integer ranging from 1 to 10;
c is an integer ranging from 5 to 50;
d is an integer ranging from 1 to 10; and
e is an integer ranging from 5 to 50;

e) from 0% to 10% by weight of at least one hydroxylated non-ionic vinylic monomer;

f) from 0% to 5% by weight of at least one cross-linking monomer;

g) from 0% to 10% by weight of at least one chain transfer agent; and

h) from 0% to 2% by weight of at least one polymeric stabilizing agent.

In one embodiment of the present disclosure, the at least one cationic polymer (i) is chosen from polymers derived from polymerizing the following mixture of monomers:
a di(C₁-C₄ alkyl)amino(C₁-C₆ alkyl)methacrylate;
at least one ester of C₁-C₃₀ alkyl and (meth)acrylic acid;
a C₁₀-C₃₀ alkyl methacrylate polyethoxylated comprising from 20 to 30 moles of ethylene oxide;
an allyl ether of polyethylene glycol/polypropylene glycol, 30/5;
a hydroxy(C₂-C₆ alkyl)methacrylate; and
an ethylene glycol dimethacrylate.

A non-limiting example of the at least one cationic polymer (i) used in the compositions of the disclosure includes the compound sold by NOVEON under the trade name AQUA CC and which corresponds to the designation INCI POLYACRYLATE-1 CROSSPOLYMER

POLYACRYLATE-1 CROSSPOLYMER is the polymerization product of a mixture of monomers comprising:
a di(C₁-C₄ alkyl)amino(C₁-C₆ alkyl)methacrylate;
at least one ester of C₁-C₃₀ alkyl and (meth)acrylic acid;
a polyethoxylated C₁₀-C₃₀ alkyl methacrylate (20-25 moles of ethylene oxide motif);
an allyl ether of polyethylene glycol/polypropylene glycol, 30/5;
a hydroxy(C₂-C₆ alkyl)methacrylate; and
an ethylene glycol dimethacrylate.

The at least one cationic polymer or polymers (i) used in the compositions of the disclosure may be present in an amount ranging from 0.01% to 10% by weight, such as 0.05% to 5% by weight, and further such as 0.1% to 1% by weight relative to the total composition weight.

The at least one cationic polymer (i) used in the composition of the disclosure may be prepared using conventional polymerization techniques such as emulsion polymerization. Polymerization may be carried out by a simple discontinuous process, by a controlled addition process, or the reaction may be initiated in a small reactor then the mass of monomers may be added in a controlled manner to the reactor (seeding process). For example, polymerization may be carried out at a reaction temperature ranging from 20° C. to 80° C., although higher or lower temperatures may be employed. To facilitate emulsification of the mixture of monomers, the emulsion polymerization is carried out in the presence of a surfactant present in a quantity of 1% to 10% by weight, such as 3% to 8% by weight, and further such as 5% to 7% by weight, relative to the total weight of the emulsion. The emulsion polymerization reaction medium also comprises at least one radical initiator, for example, present in an amount ranging from 0.01% to 3% by weight relative to the total weight of the mixture of monomers. Polymerization may be carried out in an aqueous medium or a hydroalcoholic medium at a neutral or slightly alkaline pH.

In a typical polymerization, the mixture of monomers is added, with stirring, to a solution of emulsifying surfactants such as a non-ionic surfactant, such as a linear or branched alcohol ethoxylate or a mixture of non-ionic and anionic surfactants, such as fatty alcohol sulfates or fatty alcohol alkylsulfonates, in a suitable quantity of water, in a suitable reactor, to prepare the emulsion of monomers. The emulsion is deoxygenated using any known method, then the polymerization reaction is initiated by adding a polymerization catalyst (initiator) such as sodium persulfate, or any other suitable addition polymerization catalyst, as is well known in the polymer field. The reaction mixture is stirred until polymerization is complete, for example for a period of 4 hours to 16 hours. The emulsion of monomers may be heated to a temperature ranging from 20° C. to 80° C. before adding the initiator, if desired. The quantity of monomers which has not reacted may be eliminated by adding a supplemental quantity of catalyst. The emulsion of polymer obtained may be withdrawn from the reactor and packaged for storage or use. Optionally, the pH or other physical or chemical characteristics of the emulsion may be adjusted before removing the emulsion from the reactor. The emulsion produced may have a total solids content of between 10% to 40% by weight. The total quantity of polymers in the emulsion obtained can range in an amount from 15% and 35% by weight, and, for example, not more than 25% by weight.

Suitable surfactants for facilitating emulsion polymerization include, but are not limited to, surfactants conventionally used in emulsion polymerizations, such as non-ionic, anionic, amphoteric, cationic surfactants, and mixtures thereof. In at least one embodiment, non-ionic, anionic surfactants, and mixtures thereof are used.

The polymerization may be carried out in the presence of at least one initiator resulting in the formation of free radicals. These may be chosen from, for example, insoluble inorganic persulfates such as ammonium persulfate, potassium persulfate or sodium persulfate; peroxides such as hydrogen peroxide, benzoyl peroxide, acetyl peroxide or lauryl peroxide; organic hydroperoxides such as cumene hydroperoxide or t-butyl hydroperoxide; organic peracids such as peracetic acid; and agents producing free radicals which are soluble in oil, such as 2,2′-azobis-isobutyronitrile, and mixtures thereof. The peroxides and peracids may optionally be activated with reducing agents such as sodium bisulfite or ascorbic acid, transition metals, or hydrazine, Suitable free radical initiators include, by way of non-limiting example, azo polymerization initiators which are soluble in water, such as 2,2′-azo-bis(tert-alkyl) compounds containing a hydrosolubilizing substituent on the alkyl group. The azo polymerization catalysts include, for example, the VAZOQR free radical initiators sold by DuPont, such as VAZO®44 (2,2′-azobis(2-4,5-dihydroimidazolyl)propane), VAZO®56 (2,2′-azobis(2-methylpropionamidine)dihydrochloride), and VAZO®68 (4,4′-azobis(4-cyanovaleric acid)).

In one embodiment, the compositions of the present disclosure comprise at least one ester of a C₈-C₂₄ fatty acid and oxyethylenated sorbitan comprising 2 to 10 oxyethylene motifs.

In another embodiment, the compositions of the present disclosure comprise at least one esters of a C₈-C₁₄ fatty acid and oxyethylenated sorbitan comprising 2 to 10 oxyethylene motifs.

In one embodiment, the compositions of the present disclosure comprise at least one ester of a C₁₂ fatty acid and oxyethylenated sorbitan comprising 2 to 10 oxyethylene motifs, such as 4 oxyethylene motifs.

The compositions of the present disclosure may also comprise oxyethylenated sorbitan mono-laurate containing 40E. This compound is also known as polysorbate 21. It is sold under the designation TWEEN 21 by UNIQEMA.

The compositions of the present disclosure may comprise at least 0.5% by weight of an ester of a C₈ to C₂₄ fatty acid and oxyethylenated sorbitan comprising 2 to 10 oxyethylene units, with respect to the total composition weight. For example, it comprises 0.5% to 10% by weight of an ester of a C₈ to C₂₄ fatty acid and oxyethylenated sorbitan comprising 2 to 10 oxyethylene motifs, such as 0.5% to 9% by weight, or 0.5% to 8% by weight, with respect to the total composition weight.

The compositions of the present disclosure may also contain at least one pearlizing agent.

The term “pearlizing agent” or “pearlization agent” means an agent which produces a pearlized, iridescent, moiré or metallized appearance or effect.

In one example, the pearlizing agent is a cyclodextrin.

The term “cyclodextrin” as used in the present disclosure means a cyclodextrin which has not been chemically modified or a cyclodextrin derivative corresponding to chemical modification of a cyclodextrin.

In one embodiment, cyclodextrins which may be used as a pearlizing agent in the compositions of the present disclosure are oligosaccharides with formula:

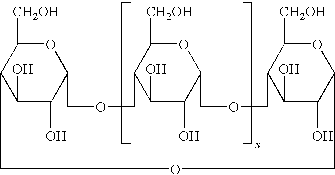

wherein x is an integer chosen from 4 (which corresponds to α-cyclodextrin), 5 (β-cyclodextrin) and 6 (γ-cyclodextrin).

The cyclodextrin may be chosen, by way of non-limiting example, from β-cyclodextrin and γ-cyclodextrin.

For example, it is possible to use a beta-cyclodextrin sold by WACKER under the designation CAVAMAX W7 PHARMA and a gamma cyclodextrin sold by WACKER under the designation CAVAMAX W8.

Non-limiting examples of cyclodextrin derivatives are methyl cyclodextrins such as methyl-beta-cyclodextrin sold by WACKER under the designation CAVASOL W7.

In accordance with the disclosure, the cyclodextrin or cyclodextrins may be present in an amount ranging from 1% to 15% by weight, such as 1% to 10% by weight and such as 1.5% to 5% by weight relative to the total composition weight.

The compositions of the disclosure may be in the form of shampoos or compositions to be applied before or after shampooing, these compositions may be in the form of a lotion which may or may not be thickened, a gel or an emulsion.

The compositions of the disclosure may thus comprise at least one surfactant chosen from anionic, cationic, amphoteric and non-ionic surfactants which are different from oxyethylenated sorbitan mono-laurate comprising 2 to 10 oxyethylene units.

Non-limiting examples of anionic surfactants which may be used in the compositions of the disclosure include salts, such as alkali metal salts such as sodium salts, ammonium salts, amine salts, aminoalcohol salts or alkaline-earth metal salts, for example magnesium salts, of the following types: alkylsulfates, alkylethersulfates, alkylamidoethersulfates, alkylarylpolyethersulfates, monoglyceride-sulfates, alkylsulfonates, alkylamidesulfonates, alkylarylsulfonates, α-olefinsulfonates, paraffinsulfonates, alkylsulfosuccinates, alkylethersulfosuccinates, alkylamide-sulfosuccinates, alkylsulfoacetates, acylsarcosinates and acylglutamates, the alkyl and acyl groups of all of these compounds containing 6 to 24 carbon atoms and the aryl group may be, for example, a phenyl or benzyl group.

It is also possible to use mono-esters of C₆-C₂₄ alkyl and polyglycoside dicarboxylic acids such as alkyl glucoside citrates, alkyl polyglycoside tartrates and alkyl polyglycoside sulfosuccinates, alkylsulfosuccinamates, acylisethionates and N-acyltaurates, the alkyl or acyl group of all of these compounds comprising 12 to 20 carbon atoms.

Another group of anionic surfactant agents which may be used in the compositions of the present disclosure, for example, includes acyl lactates the acyl group of which comprises 8 to 20 carbon atoms.

Further, alkyl-D-galactoside-uronic acids and their salts that may be mentioned include, by way of non-limiting example, polyoxyalkylenated (C₆₋₂₄ alkyl)ether-carboxylic acids, polyoxyalkylenated (C₆₋₂₄ alkyl)(C₆₋₂₄ aryl)ether-carboxylic acids, polyoxyalkylenated (C₆₋₂₄ alkyl)amidoether-carboxylic acids and their salts, such as those comprising 2 to 50 ethylene oxide motifs, and mixtures thereof.

For example, alkylsulfates, alkylethersulfates and mixtures thereof are used, such as in the form of alkali metal, alkaline-earth metal, ammonium, amine or aminoalcohol salts.

The quantity of the at least one anionic surfactant may be present in an amount ranging from 0.1% to 50% by weight, such as 4% to 20% by weight relative to the total composition weight.

Non-limiting examples of additional non-ionic surfactants which may be used in the compositions of the present disclosure have been described, for example, in the “Handbook of Surfactants” by M. R. PORTER, Blackie & Son publishers (Glasgow and London), 1991, pp 116-178. For example, the surfactants may be chosen from alcohols, alpha-diols, (C₁₋₂₀ alkyl)phenols and polyethoxylated, polypropoxylated and polyglycerolated fatty acids having a fatty chain comprising 8 to 18 carbon atoms, for example, the number of possible ethylene oxide or propylene oxide groups in particular comprising from 2 to 50 and the number of possible glycerol groups comprising, for example, from 2 to 30.

Other non-limiting examples include condensates of ethylene oxide and propylene oxide on fatty alcohols; polyethoxylated fatty acids, comprising, for example, 2 to 30 ethylene oxide motifs, polyglycerolated fatty amides comprising an average of 1 to 5 glycerol groups and, for example, 1.5 to 4, fatty acid esters of ethoxylated sorbitan comprising 2 to 30 ethylene oxide motifs, fatty acid esters of saccharose, fatty acid esters of polyethylene glycol, (C₆₋₂₄ alkyl)polyglycosides, N—(C₆₋₂₄ alkyl)glucamine derivatives, amine oxides such as oxides of (C₁₀₋₁₄)amines or oxides of N—(C₁₀₋₁₄ acyl)aminopropylmorpholine.

The additional non-ionic surfactants may be present in an amount ranging from 0.01% to 20% by weight, such as 0.2% to 10% by weight relative to the total composition weight.

The amphoteric or zwitterionic surfactant agents which may be used in the present disclosure include, for example, derivatives of secondary or tertiary aliphatic amines in which the aliphatic group is a linear or branched chain comprising 8 to 22 carbon atoms and comprising at least one anionic group such as a carboxylate, sulfonate, sulfate, phosphate or phosphonate group, for example. Non-limiting examples which may be cited are (C₈₋₂₀ alkyl)betaines, sulfobetaines, (C₈₋₂₀ alkyl)amido(C₆₋₈ alkyl)betaines or (C₈₋₂₀ alkyl)amido(C₆₋₈ alkyl)sulfobetaines.

Amine derivatives which may be mentioned include products sold under the trade name MIRANOL®, as described in U.S. Pat. Nos. 2,528,378 and 2,781,354 and classified in the CTFA dictionary, 3rd edition, 1982, with designations Amphocarboxyglycinate and Amphocarboxypropionate with respective structures (VI) and (VII):

Rₐ—CONHCH₂CH₂—N(R_{b})(R_{c})(CH₂COO⁻) (VI)

wherein:
Rₐ is an alkyl group derived from an acid Rₐ—COOH present in hydrolysed coprah oil, a heptyl group, a nonyl group or an undecyl group;
R_{b} is a beta-hydroxyethyl group; and
R_{c} is a carboxymethyl group;
and

Rₐ′—CONHCH₂CH₂—N(B)(B′) (VII)

wherein:
B is —CH₂CH₂OX′;
B′ is —(CH₂)_{z}—Y′, where z is an integer ranging from 1 to 2;
X′ is chosen from a —CH₂CH₂—COOH group and a hydrogen atom;
Y′ is chosen from a —COOH and a —CH₂—CHOH—SO₃H;
Rₐ′ is an alkyl group of an acid Rₐ′—COOH present in coprah oil or in hydrolysed linseed oil, an alkyl group, for example, C₁₇ and its iso form, or an unsaturated C₁₇ group.

These compounds have been classified in the CTFA dictionary, 5th edition, 1993 under the designations disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, and cocoamphodipropionic acid.

An example which may be cited is the cocoamphodiacetate sold by RHODIA under the trade name MIRANOL® C2M concentrate.

Non-limiting examples of amphoteric or zwitterionic surfactants of those cited above include (C₈₋₂₀ alkyl)betaines, (C₈₋₂₀ alkyl)amido(C₆₋₈ alkyl)betaines and mixtures thereof.

The quantity of the at least one amphoteric or zwitterionic surfactant may be present in an amount ranging from 0.01% to 20% by weight, such as in the range 0.5% to 10% by weight relative to the total composition weight.

In one embodiment, the compositions of the present disclosure comprise at least one anionic surfactant and at least one amphoteric or zwitterionic surfactant.

The compositions of the present disclosure has, for example, a total anionic, non-ionic, amphoteric or zwitterionic surfactant content in an amount ranging from 4% to 50% by weight, such as in the range 4% to 20% by weight relative to the total composition weight.

In another embodiment, the cosmetic compositions of the disclosure further comprise at least one cationic surfactant.

Non-limiting examples of a cationic surfactant which may in particular be cited are salts of primary, secondary or tertiary fatty amines, optionally polyoxyalkylenated; quaternary ammonium salts such as tetraalkyl ammonium, alkylamidoalkyltrialkyl ammonium, trialkylbenzyl ammonium, trialkylhydroxylalkyl ammonium or alkylpyridinium chlorides or bromides; imidazoline derivatives; or oxides of amines with a cationic nature; and quaternized diesters.

The at least one cationic surfactant may be present in an amount ranging from 0.01% to 10% by weight, such as 0.2% to 5% by weight, and further such as 0.3% to 3% by weight relative to the total weight of the cosmetic composition.

The compositions of the present disclosure may further comprise at least one cationic polymer which is different from the at least one cationic polymer (i).

The term “cationic polymer” means any polymer containing cationic groups and/or groups which can be ionized to cationic groups.

In one embodiment, the at least one cationic polymer which can be used in the present disclosure may be chosen from those known to improve the cosmetic properties of treated hair, for example those described in patent application EP-A-0 337 354 and in French patent applications FR-A-2 270 846, FR-A-2 383 660, FR-A-2 598 611, FR-A-2 470 596 and FR-A-2 519 863.

Non-limiting examples of the at least one cationic polymer includes those which comprise motifs comprising primary, secondary, tertiary and/or quaternary amine groups which may either form part of the principal polymer chain or be carried by a side chain substituent directly bonded thereto.

The at least one cationic polymer may have a mass average molecular mass of more than 10⁵, such as more than 10⁶ and further such as in the range of 10⁶ to 10⁸.

Non-limiting examples of the at least one cationic polymer includes polymers of the polyamine, polyaminoamide and quaternary polyammonium type.

Non-limiting examples of polymers of the polyamine, polyaminoamide and quaternary polyammonium type which may be used in the present disclosure include those described in French patents FR-A-2 505 348 and FR-A-2 542 997. The following of these polymers may be mentioned, for example:

(1) Homopolymers or copolymers derived from acrylic or methacrylic esters or amides and comprising at least one of the following motifs with formulae (VI), (VII), (VIII) or (IX):

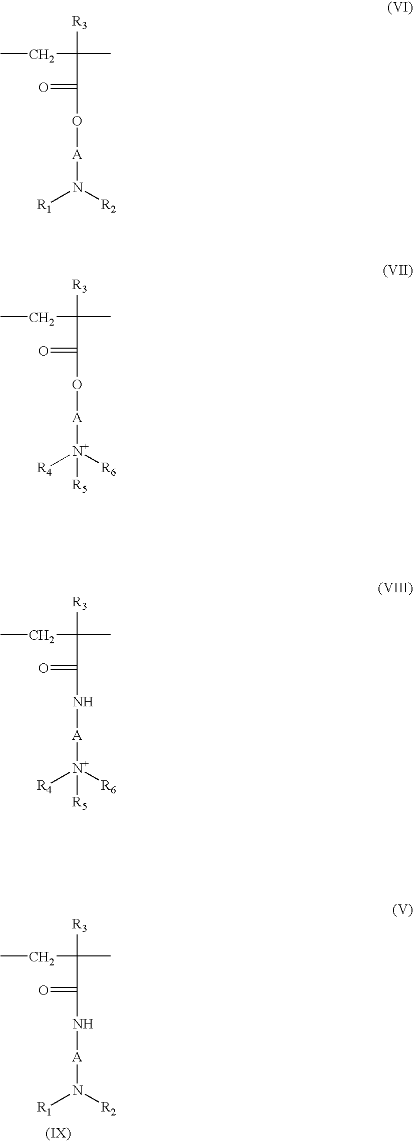

wherein:
R₃, which may be identical or different, is chosen from a hydrogen atom and a radical CH₃;
A, which may be identical or different, is chosen from a linear and branched alkyl group comprising 1 to 6 carbon atoms, such as 2 or 3 carbon atoms and a hydroxyalkyl group comprising 1 to 4 carbon atoms;
R₄, R₅ and R₆, which may be identical or different, are chosen from alkyl groups comprising 1 to 18 carbon atoms and benzyl radicals, such as alkyl groups comprising 1 to 6 carbon atoms;
R₁ and R₂, which may be identical or different, are chosen from hydrogen atoms and alkyl groups comprising 1 to 6 carbon atoms, such as methyl or ethyl group;
X is an anion derived from a mineral or organic acid such as a methosulfate anion or a halide such as a chloride or bromide.

The polymers in family (1) may also comprise at least one motif derived from co-monomers which may be chosen from, by way of non-limiting example, acrylamides, methacrylamides, diacetone acrylamides, acrylamides and methacrylamides substituted on the nitrogen by lower alkyls (C₁-C₄), acrylic or methacrylic acids or their esters, vinyllactames such as vinylpyrrolidone or vinylcaprolactame, or vinyl esters.

Polymers of family (1) which may thus be mentioned, by way of non-limiting example, include:

copolymers of acrylamide and dimethylaminoethyl methacrylate quaternized with dimethyl sulfate or with a dimethyl halide;

copolymers of acrylamide and methacryloyloxyethyltrimethyl ammonium chloride described, for example, in European patent application EP-A-080 976;

the copolymer of acrylamide and methacryloyloxyethyltrimethyl ammonium methosulfate;

vinylpyrrolidone/dialkylaminoalkyl acrylate or methacrylate copolymers, which may or may not be quaternized. Such polymers are described in detail in French patents FR-A-2 077 143 and FR-A-2 393 573;

dimethyl aminoethyl methacrylate/vinylcaprolactam/vinylpyrrolidone terpolymers;

vinylpyrrolidone/methacrylamidopropyl dimethylamine copolymers;

quaternized vinylpyrrolidone/dimethylaminopropyl methacrylamide copolymers;

cross-linked polymers of methacryloyloxyalkyl(C₁-C₄)trialkyl(C₁-C₄)ammonium salts such as the polymers obtained by homopolymerization of dimethylaminoethylmethacrylate quaternized with methyl chloride or by copolymerization of acrylamide with dimethylaminoethylmethacrylate quaternized with methyl chloride, homo- or copolymerization being followed by cross-linking by a compound containing an olefinically unsaturated bond, such as methylene bis-acrylamide. In another embodiment, it is possible to use a cross-linked acrylamide/methacryloyloxyethyltrimethyl ammonium chloride copolymer (20/80 by weight) in the form of a dispersion containing 50% by weight of said copolymer in mineral oil. This dispersion is sold under the trade name “SALCARE® SC 92” by CIBA. It is also possible to use a cross-linked homopolymer of methacryloyloxyethyltrimethyl ammonium chloride containing about 50% by weight of homopolymer in mineral oil or in a liquid ester. These dispersions are sold under the trade names “SALCARE® SC 95” and “SALCARE® SC 96” by CIBA.

(2) Cationic polysaccharides, for example those chosen from:

(a) cellulose ether derivatives comprising quaternary ammonium groups described in French patent FR-A-1 492 597. These polymers are also defined in the CTFA dictionary as quaternary ammonium hydroxycelluloses which have reacted with an epoxide substituted with a trimethyl ammonium group;

(b) copolymers of cellulose or cellulose derivatives grafted with a hydrosoluble quaternary ammonium monomer and described in particular in U.S. Pat. No. 4,131,576, such as hydroxyalkylcelluloses, for example hydroxymethyl-, hydroxyethyl- or hydroxypropylcelluloses for example grafted with a methacryloylethyl trimethylammonium, methacrylamidopropyl trimethylammonium, or dimethyl-diallylammonium salt;

(c) cationic polygalactomannanes, such as described in U.S. Pat. Nos. 3,589,578 and 4,031,307, such as guar gums comprising trialkylammonium groups. An example of a guar gum modified by a salt (for example chloride) which may be used is 2,3-epoxypropyl trimethylammonium.

(3) Polymers constituted by piperazinyl motifs and divalent alkylene or hydroxyalkylene radicals with straight or branched chains, optionally interrupted by oxygen, sulfur or nitrogen atoms or by aromatic or heterocyclic cycles, as well as the oxidation products and/or quaternization products of said polymers.

Such polymers have been described, for example, in French patents FR-A-2 162 025 and FR-A-2 280 361.

(4) Water-soluble polyaminoamides, for example prepared by polycondensation of an acidic compound with a polyamine; said polyaminoamides may be cross-linked by an epihalohydrin, a diepoxy compound, a dianhydride, an unsaturated dianhydride, a bis-unsaturated derivative, a bis-halohydrin, a bis-azetidinium compound, a bis-haloacyidiamine, an alkyl bis-halide or by an oligomer resulting from the reaction of a bi-functional compound with a bis-halohydrin, a bis-azetidinium, a bis-haloacyldiamine, an alkyl bis-halide, an epilhalohydrin, a diepoxide or a bis-unsaturated derivative; the cross-linking agent being used in proportions of 0.025 to 0.35 moles per amine group of the polyaminoamide; said polyaminoamides may be alkoylated or, if they comprise at least one tertiary amine function, they may be quaternized. Such polymers have in particular been described in French patents FR-A-2 252 840 and FR-A-2 368 508;

(5) Polyaminoamide derivatives resulting from condensation of polyalkoylene polyamines with polycarboxylic acids followed by alkoylation with bi-functional agents. Non-limiting examples which may be cited are adipic acid -diacoylaminohydroxyalkoyidialkoylene triamine in which the alkoyl radical comprises 1 to 4 carbon atoms such as methyl, ethyl, or propyl radicals. Such polymers have been described, for example, in French patent FR-A-1 583 363.

Non-limiting examples of these derivatives which may be mentioned are adipic acid/dimethylaminohydroxypropyl/diethylenetriamine polymers.

(6) Polymers obtained by reaction of a polyalkylene polyamine comprising two primary amine groups and at least one secondary amine group with a dicarboxylic acid selected from diglycolic acid and saturated aliphatic dicarboxylic acids comprising 3 to 8 carbon atoms. Since the molar ratio between the polyalkylene polyamine and the dicarboxylic acid is in the range 0.8:1 to 1.4:1, the resulting polyaminoamide can be reacted with epichlorhydrin in a molar ratio of epichlorhydrin with respect to the secondary amine group of the polyaminoamide in the range 0.5:1 to 1.8:1. Such polymers have been described in, for example, U.S. Pat. Nos. 3,227,615 and 2,961,347.

(7) Alkyldiallylamine or dialkyldiallyl ammonium cyclopolymers such as homopolymers or copolymers comprising, as the principal constituents of the chain, motifs with formulae (X) or (XI):

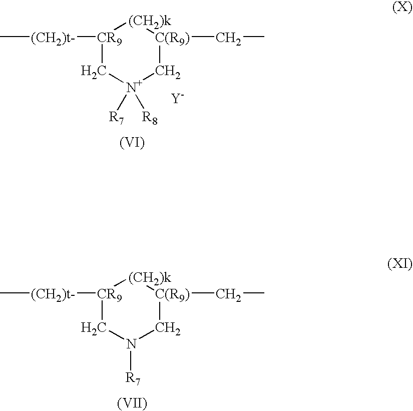

wherein k and t are integers ranging from 0 to 1, the sum k+t being equal to 1; R₉ is chosen from a hydrogen atom or a methyl radical; R₇ and R₈, which are independent of each other, are chosen from alkyl groups comprising 1 to 6 carbon atoms, hydroxyalkyl groups in which the alkyl group comprises 1 to 5 carbon atoms, lower (C₁-C₄) amidoalkyl groups, or R₇ and R₈ may together with the nitrogen atom to which they are attached form heterocylic groups such as piperidinyl or morpholinyl; R₇ and R₅ independently are chosen from alkyl groups comprising 1 to 4 carbon atoms; Y⁻ is an anion such as bromide, chloride, acetate, borate, citrate, tartrate, bisulfate, bisulfite, sulfate or phosphate. These polymers have been described, for example, in French patent FR-A-2 080 759 and in its patent of addition, FR-A-2 190 406;

(8) The quaternary diammonium polymer comprising repeat motifs with formula:

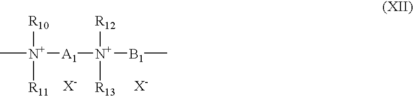

wherein:

R₁₀, R₁₁, R₁₂ and R₁₃, which may be identical or different, are chosen from aliphatic, alicyclic, or arylaliphatic radicals comprising 1 to 20 carbon atoms or lower hydroxyalkylaliphatic radicals, or R₁₀, R₁₁, R₁₂ and R₁₃, together or separately constitute, with the nitrogen atoms to which they are attached, heterocycles optionally comprising a second heteroatom other than nitrogen, or R₁₀, R₁₁, R₁₂ and R₁₃ are chosen from linear and branched C₁-C₆ alkyl radicals substituted with a nitrile, ester, acyl, amide and —CO—O—R₁₄-D and —CO—NH—R₁₄-D, wherein R₁₄ is an alkylene and D is a quaternary ammonium group;

A₁ and B₁, which may be identical or different, are chosen from polymethylene groups comprising 2 to 20 carbon atoms which may be linear or branched, saturated or unsaturated and may comprise, bonded to or interposed in the principal chain, at least one aromatic cycle, or at least one oxygen or sulfur atom, or sulfoxide, sulfone, disulfide, amino, alkylamino, hydroxyl, quaternary ammonium, ureido, amide or ester groups; and

X⁻ is an anion derived from a mineral or organic acid;

A₁, R₁₀ and R₁₂ may, with the two nitrogen atoms to which they are attached, form a piperazine cycle; further, if A₁ is a linear or branched, saturated or unsaturated alkylene or hydroxyalkylene radical, B₁ may be a —(CH₂)ₙ—CO-D-OC—(CH₂)ₙ— group wherein n is an integer ranging from 1 to 100, such as 1 to 50, and D is chosen from:

a) a glycol residue with formula: —O-Z-O—, wherein Z is chosen from a linear and branched hydrocarbon radical and a group chosen from one of the following formulae:

—(CH₂—CH₂—O)x-CH₂—CH₂—, and

—[CH₂—CH(CH₃)—O]y-CH₂—CH(CH₃)—

wherein x and y are integers ranging from 1 to 4, representing a defined and unique degree of polymerization or any integer ranging from 1 to 4 representing a mean degree of polymerization;

b) a bis-secondary diamine residue such as a piperazine derivative;

c) a bis-primary diamine residue with formula: —NH—Y—NH—, wherein Y is chosen from a linear and branched hydrocarbon radical and the bivalent radical:

—CH₂—CH₂—S—S—CH₂—CH₂—;

d) a ureylene group with formula: —NH—CO—NH—.

For example, X⁻ is an anion such as chloride or bromide.

These polymers have a number average molecular mass which is, for example, in the range 1000 to 100000.

Polymers of this type have been described, for example, in French patents FR-A-2 320 330, FR-A-2 270 846, FR-A-2 316 271, FR-A-2 336 434 and FR-2 413 907 and U.S. Pat. Nos. 2,273,780, 2,375,853, 2,388,614, 2,454,547, 3,206,462, 2,261,002, 2,271,378, 3,874,870, 4,001,432; 3,929,990, 3,966,904, 4,005,193, 4,025,617, 4,025,627, 4,025,653, 4,026,945 and 4,027,020.

For example, polymers which are constituted by repeat motifs with formula (XIII) below may be used:

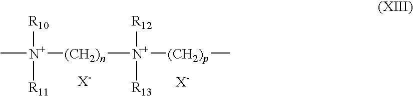

in which R₁₀, R₁₁, R₁₂ and R₁₃, which may be identical or different, are chosen from alkyl or hydroxyalkyl radicals comprising 1 to 4 carbon atoms, n and p are integers ranging from 2 to 20 and X⁻ is an anion derived from a mineral or organic acid;

(9) Poly(quaternary ammonium) polymers constituted by repeat motifs with formula (XIV):

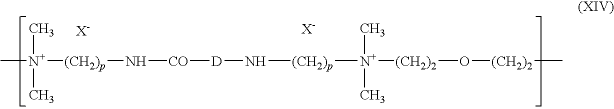

wherein p is an integer ranging from 1 to 6, D may be zero or may represent a —(CH₂)ᵣ—CO— group in which r is an integer chosen from 4 and 7, and X⁻ is an anion.

Such polymers may be prepared using the processes described in U.S. Pat. Nos. 4,157,388, 4,702,906, 4,719,282. For example, they have been described in EP-A-122 324;

(10) Quaternary vinylpyrrolidone and vinylimidazole polymers.

(11) Polyamines such as the product termed “POLYETHYLENEGLYCOL (15) TALLOW POLYAMINE” in the CTFA dictionary.

Other cationic polymers which may be used in the context of the disclosure include, by way of non-limiting example, cationic proteins or cationic protein hydrolysates, polyalkyleneimines, in particular polyethyleneimines, polymers containing vinylpyridine or vinylpyridinium motifs, condensates of polyamines and epichlorhydrin, quaternary polyureylenes and chitin derivatives.

In another embodiment of the present disclosure, the at least one cationic polymer which may be used includes cellulose ether derivatives, for example, comprising quaternary ammonium groups such as the products sold with designation “JR 400” by AMERCHOL, cationic cyclopolymers, for example, homopolymers or copolymers of dimethyldiallylammonium chloride sold under the designations MERQUAT® 100, MERQUAT® 550 and MERQUAT® S by NALCO, guar gums modified by a 2,3-epoxypropyl-trimethylammonium salt, quaternary polymers of vinylpyrrolidone and vinylimidazole and cross-linked polymers of cross-linked methacryloyloxyalkyl(C₁-C₄)trialkyl(C₁-C₄)ammonium salts.

the at least one cationic polymer may be present in an amount ranging from 0.01% to 10% by weight, such as 0.02% to 5% by weight and such as 0.05% to 1% by weight relative to the total composition weight.

The composition of the disclosure may also comprise at least one silicone.

The at least one silicone which can be used in the context of the disclosure may be, by way of non-limiting example, volatile or otherwise, soluble or insoluble in the composition. For example, they may be polyorganosiloxanes which are insoluble in the composition of the disclosure and in the form of oils, waxes, resins or gums.

The insoluble silicones are for example dispersed in the compositions in the form of particles which may have, for example, a number average size in the range 2 nanometres to 100 micrometres, such as in the range 20 nanometres to 20 micrometres (measured using a granulometer).

The polyorganosiloxanes are defined in more detail in the work by Walter NOLL, “Chemistry and Technology of Silicones” (1968) Academic Press. They may be volatiles or non-volatiles.

When they are volatile, the at least one silicone is chosen from, for example, those with a boiling point in the range 60° C. to 260° C., such as from:

(i) cyclic silicones comprising 3 to 7 silicon atoms, such as 4 or 5 silicon atoms. It may, for example, be octamethylcyclotetra-siloxane sold under the trade name “VOLATILE SILICONE 7207” by UNION CARBIDE or “SILBIONE 70045 V 2” from RHODIA, decamethylcyclopentasiloxane sold under the trade name “VOLATILE SILICONE 7158” by UNION CARBIDE, or “SILBIONE 70045 V 5” from RHODIA, and mixtures thereof.

Cyclocopolymers of the dimethyl-siloxane/methylalkylsiloxane type may also be cited, such as “SILICONE VOLATILE FZ 3109” sold by UNION CARBIDE, with chemical structure:

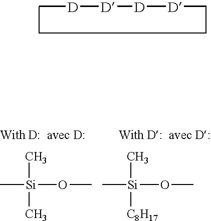

Mixtures of cyclic silicones with organic compounds derived from silicon may also be cited, such as the mixture of octamethylcyclotetrasiloxane and tetratrimethylsilylpentaerythritol (50/50) and the mixture of octamethylcyclotetrasiloxane and oxy-111′-(hexa-2,2,2′,2′,3,3′-trimethylsilyloxy)bis-neopentane;

(ii) Linear volatile silicones comprising 2 to 9 silicon atoms and having a viscosity of 5×10⁻⁶ m²/s or less at 25° C. Non-limiting examples include decamethyltetrasiloxane sold in particular under the designation “SH 200” by TORAY SILICONE. Silicones falling within this category are also described in the article published in Cosmetics and Toiletries, Vol. 91, Jan. 76, p. 27-32—TODD & BYERS “Volatile silicone fluids for cosmetics”.

Non-limiting examples of non-volatile silicones include polyalkylsiloxanes, polyarylsiloxanes, polyalkylarylsiloxanes, silicone resins and gums, polyorganosiloxanes modified by organofunctional groups, linear polysiloxane(A)-polyoxyalkylene(B) block copolymers of the (A-B)ₙ type where n>3; graft silicone polymers with a non-silicone organic backbone constituted by a principal organic chain formed by organic monomers containing no silicone onto which are grafted, within said chain and optionally at one of its ends at least, at least one polysiloxane macromonomer; grafted silicone polymers with a polysiloxane backbone grafted with non-silicone organic monomers, comprising a principal polysiloxane chain onto which is grafted, within said chain and optionally on one of its ends at least, at least one organic macro monomer comprising no silicone; and mixtures thereof.

Non-limiting examples of polyalkylsiloxanes include polydimethylsiloxanes with trimethylsilyl terminal groups having a viscosity of 5×10⁻⁶ to 2.5 m²/s at 25° C., such as 1×10⁻⁵ to 1 m²/s. The viscosity of the silicones is, for example, measured at 25° C. in accordance with American standard ASTM 445 Appendix C.

Non-limiting examples of these polyalkylsiloxanes which may be cited include the following commercial products:

SILBIONE oils from series 47 and 70 047 or MIRASIL oils sold by Rhodia Chimie, such as 70 047 V 500 000 oil;

Oils from the MIRASIL series sold by Rhodia Chimie;

Oils from series 200 from DOW CORNING, more particular DC200 with a viscosity of 60000 cSt;

VISCASIL oils from GENERAL ELECTRIC and certain oils from the SF series (SF 96, SF 18) from GENERAL ELECTRIC.

Polydimethylsiloxanes may have terminal dimethylsilanol groups (Dimethiconol according to the CTFA designation), such as oils from the 48 series from Rhodia Chimie.

Polyalkylsiloxanes may include commercial products sold under the denominations “ABIL WAX 9800 and 9801” by GOLDSCHMIDT; they are polyalkyl (C₁-C₂₀) siloxanes.

The polyalkylarylsiloxanes may, for example, be chosen from polydimethyl-methylphenylsiloxanes and linear and/or branched polydimethyl-diphenylsiloxanes with a viscosity of 1×10⁻⁵ to 5×10⁻² m²/s at 25° C.

Non-limiting examples of these polyalkylarylsiloxanes include the products sold under the following denominations:

SILBIONE oils from the 70 641 series from Rhodia Chimie;

oils from the RHODORSIL 70 633 and 763 series from Rhodia Chimie;

DOW CORNING 556 COSMETIC GRADE FLUID oil from DOW CORNING;

silicones from the PK series from BAYER, such as the PK20 product;

silicones from the PN, PH series from BAYER, such as the products PN1000 and PH1000;

certain oils from the SF series from GENERAL ELECTRIC, such as SF 1023, SF 1154, SF 1250 or SF 1265.

Non-limiting examples of silicone gums which can be used in accordance with the disclosure include polydiorganosiloxanes with high mass average molecular masses in the range 200000 to 1000000, used alone or as a mixture in a solvent. Said solvent may be chosen from volatile silicones, polydimethylsiloxane oils (PDMS), polyphenylmethylsiloxane oils (PPMS), isoparaffins, polyisobutylenes, methylene chloride, pentane, dodecane, tridecanes and mixtures thereof.

In another embodiment, the following products can be mentioned, by way of non-limiting example:

polydimethylsiloxane gums;

polydimethylsiloxane/methylvinylsiloxane gums;

polydimethylsiloxane/diphenylsiloxane gums;

polydimethylsiloxane/phenylmethylsiloxane gums;

polydimethylsiloxane/diphenylsiloxane/methylvinylsiloxane gums.

It is also possible to use mixtures of silicone such as:

mixtures formed from a polydimethylsiloxane gum hydroxylated at the chain end (denoted dimethiconol in the CTFA dictionary nomenclature), and a cyclic polydimethylsiloxane (denoted cyclomethicone in the CTFA dictionary) such as the product Q2 1401 sold by DOW CORNING;

mixtures formed from a polydimethylsiloxane gum with a cyclic silicone, such as the product SF 1214 Silicone Fluid from GENERAL ELECTRIC; this product is an SF 30 gum corresponding to a dimethicone having a number average molecular weight of 500000 dissolved in SF 1202 Silicone Fluid oil corresponding to decamethylcyclopentasiloxane;

mixtures of two PDMS with different viscosities, and more particularly a PDMS gum and a PDMS oil, such as the product SF 1236 from GENERAL ELECTRIC. SF 1236 is a mixture of an SE 30 gum as defined above having a viscosity of 20 m²/s and an SF 96 oil with a viscosity of 5×10⁻⁶ m²/s. This product may comprise, for example, 15% of SE 20 gum and 85% of an SF 96 oil.

Organopolysiloxane resins which can be used in accordance with the present disclosure are cross-linked siloxane systems comprising the following units: R₂SiO_{2/2}, R₃SiO_{1/2}, RSiO_{3/2} and SiO_{4/2}, in which R represents a hydrocarbon group comprising 1 to 16 carbon atoms or a phenyl group.

R represents, for example, a C₁-C₄ lower alkyl radical, for example, methyl, or a phenyl radical.

These resins include the product sold under the trade name “DOW CORNING 593” or those sold under the under the trade names “SILICONE FLUID SS 4230 and SS 4267” by GENERAL ELECTRIC; they are silicones with a dimethyl/trimethyl siloxane structure.

Trimethylsiloxysilicate resins may be sold under the trade names X22-4914, X21-5034 and X21-5037 by SHIN-ETSU.

Organomodified silicones for use in the disclosure are silicones such as those defined above and comprising in their structure at least one organofunctional groups bonded via a hydrocarbon group.

Organomodified silicone which may be cited include polyorganosiloxanes comprising:

polyethyleneoxy and/or polypropyleneoxy groups possibly comprising C₆-C₂₄ alkyl groups, such as the products denoted dimethicone-copolyol sold by DOW CORNING under the trade name DC 1248 or SILWET® L 722, L 7500, L 77, L 711 oils from UNION CARBIDE and alkyl(C₁₂)-methicone-copolyol sold by DOW CORNING under the trade name Q2 5200;

amine groups, which may or may not be substituted, such as the products sold under the trade name GP 4 Silicone Fluid and GP 7100 by GENESEE or the products sold under the trade names Q2 8220 and DOW CORNING 929 or 939 by DOW CORNING. Particular substituted amine groups are C₁-C₄ aminoalkyl groups;

quaternary ammonium groups such as the products sold under the trade names ABILQUAT 3272 and ABILQUAT 3474 by GOLDSCHMIDT;

thiol groups, such as the products sold under the trade names “GP 72 A” and “GP 71” by GENESEE;

alkoxy groups such as the product sold under the trade name “SILICONE COPOLYMER F-755” by SWS SILICONES and ABIL WAX® 2428, 2434 and 2440 by GOLDSCHMIDT;

hydroxyl groups, such as the polyorganosiloxanes with a hydroxyalkyl function described in French patent application FR-A-85 16334;

acyloxyalkyl groups such as the polyorganosiloxanes described in U.S. Pat. No. 4,957,732;

anionic carboxylic acid type groups such as those, for example, described in EP-A-186 507 from CHISSO CORPORATION, or of the alkylcarboxylic type, such as those present in the product X-22-3701 E from SHIN-ETSU; 2-hydroxyalkylsulfonate; 2-hydroxyalkylthiosulfate, such as the products sold by GOLDSCHMIDT under the trade names “ABIL® S201” and “ABIL® S255”;

hydroxyacylamino groups such as the polyorganosiloxanes described in patent application EP-A-342 834. An example which may be cited is the product Q2-8413 from DOW CORNING.

Non-limiting examples of the at least one silicone for use in the disclosure include polydimethylsiloxanes such as polydimethylsiloxanes with trimethylsilyl terminal groups, or polydimethylsiloxanes with hydroxydimethylsilyl terminal groups, and amino-containing silicones.

The at least one silicone may be present in an amount ranging from 0.05% to 20% by weight, such as 0.1% to 10% by weight, such as 0.5% to 5% by weight with respect to the total composition weight.

The term “cosmetically acceptable medium” means a medium which is compatible with keratinous material, such as the hair and skin.

The cosmetically acceptable medium is constituted by water or a mixture of water and at least one cosmetically acceptable solvent chosen from C₁-C₄ lower alcohols, such as ethanol, isopropanol, tertio-butanol or n-butanol; polyols such as glycerol, propylene glycol or polyethylene glycols; and mixtures thereof.

The pH of the compositions of the disclosure may be, for example, less than 7, such as less than 6, in the range of 2 to 6, and such as in the range of 3 to 6.

The compositions of the disclosure may also comprise at least one conventional additive which is well known in the art, such as: thickeners or viscosity regulators, which may be natural or synthetic; C₁₂-C₃₀ fatty alcohols; ceramides; oily fatty esters such as isopropyl myristate or triglycerides; mineral or synthetic oils such as α-olefins; vitamins or pro-vitamins; pH stabilizing agents; preservatives; and colorants.

The skilled person will carefully select any additives and their quantities in a manner such that they do not interfere with the properties of the compositions of the present disclosure.

The at least one additive may be present in the compositions of the disclosure in an amount ranging from 0 to 20% by weight with respect to the total composition weight.

The compositions of the disclosure may be used to wash and condition keratinous material, such as hair, for example as a shampoo, or to condition keratinous material, for example in a conditioner.

The present disclosure also provides methods for the cosmetic treatments of keratinous materials such as hair, comprising applying an effective quantity of the compositions as defined above onto said materials, and rinsing after leaving on for a period of time.

The following examples are intended to illustrate the disclosure without being limiting.

### EXAMPLE

Two conditioners in accordance with the present disclosure were prepared from the ingredients shown in the Table below.

| Compositions | A | B |
|---|---|---|
| Sodium chloride | 1 | 1 |
| Lactic acid | 0.343 | 0.286 |
| Salicylic acid (preservative) | 0.15 | 0.2 |
| Ethyl p-hydroxybenzoate (preservative) | 0.5 | 0.15 |
| Sodium benzoate (preservative) | 0.4 | 0.5 |
| Methyl p-hydroxybenzoate, sodium salt | 0.5 | 0.4 |
| (preservative) | | |
| Fragrance | 0.5 | 0.5 |
| Polyacrylate-1 Crosspolymer, 20% emulsion | 3 | 3 |
| in water ⁽¹⁾ | | |
| Hydroxyethyl cellulose quaternized with 2,3 | 0.4 | — |
| epoxypropyl trimethyl ammonium chloride ⁽²⁾ | | |
| Hydroxypropyl guar trimethyl ammonium | — | 0.2 |
| chloride ⁽³⁾ | | |
| Beta-cyclodextrin (cyclomaltoheptaose) ⁽⁴⁾ | 2 | 2 |
| Polydimethylsiloxane (viscosity 500000 cSt) ⁽⁵⁾ | 1.5 | — |
| Polydimethylsiloxane ⁽⁶⁾ (viscosity 60000 cSt) | — | 2.7 |
| Sorbitan monolaurate, oxyethylenated (4 OE) ⁽⁷⁾ | 6 | 0.2 |
| Cocoyl amidopropyl betaine in aqueous | 6.41 | 6.16 |
| solution (47% AM) ⁽⁸⁾ | | |
| Sodium lauryl ether sulfate (2.2 OE) in | 22.02 | 19.93 |
| aqueous solution (70% AM) ⁽⁹⁾ | | |
| Deionized water | Qsp 100 g | Qsp 100 g |

⁽¹⁾ sold under the trade name Carbopol Aqua CC by Noveon
⁽²⁾ sold under the trade name JR400 by Amerchol
⁽³⁾ sold under the trade name Jaguar C13S by Rhodia Chimie
⁽⁴⁾ sold under the trade name Cavamax W7 Pharma by WACKER
⁽⁵⁾ sold under the trade name Dow Corning 200 Fluid 500000 by Dow Corning
⁽⁶⁾ sold under the trade name Dow Corning 200 Fluid 60000 by Dow Corning
⁽⁷⁾ sold under the trade name Tween 21 by Uniqema,
⁽⁸⁾ sold under the trade name Tego Betaïne F50 by Goldschmidt,
⁽⁹⁾ sold under the trade name Texapon AOS 225UP by COGNIS

These compositions had a good texture which was stable with time. They were gentle on the scalp and when applied to hair washed it well and, finally, provided it with good conditioning properties.

A conditioner composition in accordance with the present disclosure was prepared using the following ingredients:

Chemical name

| | |
|---|---|
| Lactic acid | 0.343 |
| Chlorhexidine hydrochloride (preservative) | 0.02 |
| Ethyl p-hydroxybenzoate (preservative) | 0.2 |
| Lanolin | 0.15 |
| Cetyl alcohol | 2.5 |
| Fragrance | 0.4 |
| Myristyl/cetyl/stearyl myristate/palmitate/stearate | 0.25 |
| Polyacrylate-1 Crosspolymer, 20% emulsion in water ⁽¹⁾ | 2.5 |
| Hydroxyethyl cellulose ⁽²⁾ | 0.2 |
| Poly lauryl methyl/methyisiloxane, oxyethylenated (18 | 0.15 |
| OE) and oxypropylenated (18 OP) ⁽³⁾ | |
| Polydimethylsiloxane with aminoethyliminopropyl groups | 1.08 |
| with a methoxy and/or hydroxy function and alpha- | |
| omega silanols in cationic aqueous emulsion ⁽⁴⁾ | |
| Polyethylene glycol (180 OE) ⁽⁵⁾ | 2 |
| Cetyl trimethyl ammonium chloride in aqueous solution ⁽⁶⁾ | 1.8 |
| Sorbitan monolaurate, oxyethylenated (4 OE) ⁽⁷⁾ | 4 |
| Stearyl amidopropyl dimethyl amine ⁽⁸⁾ | 0.75 |
| Deionized water | 84 |

⁽¹⁾ sold under the trade name Carbopol Aqua CC by Noveon
⁽²⁾ sold under the trade name Natrosol 250 HHR by Aqualon
⁽³⁾ sold under the trade name Dow Corning 5200 by Dow Corning
⁽⁴⁾ sold under the trade name Dow Corning 2-8299 by Dow Corning
⁽⁵⁾ sold under the trade name Carbowax sentry PEG 8000 Granular NF/FCC by DOW CHEMICAL
⁽⁶⁾ sold under the trade name Arquad 16-25 by AKZO NOBEL
⁽⁷⁾ sold under the trade name Tween 21 by Uniqema
⁽⁸⁾ sold under the trade name Mackine 301 by Mackintire

This composition had a good texture which was stable over time. It was gentle on the scalp, and when applied to the hair as a conditioner, it endowed the hair with good disentangling properties.

What is claimed is:

1. A composition for the cosmetic treatment of keratinous material, comprising, in a cosmetically acceptable medium:
(i) at least one cationic polymer obtained by polymerizing a mixture of monomers comprising at least one vinylic monomer substituted with at least one amino group, at least one hydrophobic non-ionic vinylic monomer, and at least one associative vinylic monomer, and;
(ii) at least one ester of a C₈-C₂₄ fatty acid and oxyethylenated sorbitan comprising 2 to 10 oxyethylene motifs.

2. The composition according to claim 1, wherein the at least one vinylic monomer substituted with at least one amino group is chosen from:
mono(C₁-C₄ alkyl)amino(C₁-C₈ alkyl)(meth)acrylates;
di(C₁-C₄ alkyl)amino(C₁-C₈ alkyl)(meth)acrylates;
mono(C₁-C₄ alkyl)amino(C₁-C₈ alkyl)(meth)acrylamides;
di(C₁-C₄ alkyl)amino(C₁-C₈ alkyl)(meth)acrylamides;
(meth)acrylamides containing a heterocyclic group comprising a nitrogen atom;
(meth)acrylates containing a heterocyclic group comprising a nitrogen atom;
nitrogen-containing heterocycles comprising at least one vinyl group;
and mixtures thereof.

3. The composition according to claim 2, wherein the at least one vinylic monomer substituted with at least one amino group is chosen from:
2-(N,N-dimethylamino)ethyl(meth)acrylate,
3-(N,N-dimethylamino)propyl(meth)acrylate,
4-(N,N-dimethylamino)butyl(meth)acrylate,
(N,N-dimethylamino)-t-butyl(meth)acrylate,
2-(N,N-diethylamino)ethyl(meth)acrylate,
3-(N,N-diethylamino)propyl(meth)acrylate,
4-(N,N-diethylamino)butyl(meth)acrylate,
2-(N,N-dipropylamino)ethyl(meth)acrylate,
3-(N,N-dipropylamino)propyl(meth)acrylate
4-(N,N-dipropylamino)butyl(meth)acrylate,
N′-(2-N,N-dimethylamino)ethyl(meth)acrylamide,
N′-(3-N,N-dimethylamino)propyl acrylamide,
N-(2-pyridyl)acrylamide,
N-(2-imidazolyl)methacrylamide,
2-(4-morpholinyl)ethyl methacrylate,
2-(4-morpholinyl)ethyl acrylate,
N-(4-morpholinyl)methacrylamide,
N-(4-morpholinyl)acrylamide, and
2-vinylpyridine and 4-vinylpyridine.

4. The composition according to claim 1, wherein the at least one vinylic monomer substituted with at least one amino group is present in an amount ranging from 10% to 70% by weight, relative to the total weight of the mixture of monomers.

5. The composition according to claim 1, wherein the at least one hydrophobic non-ionic vinylic monomer is chosen from formulae (I) and (II):
CH₂═C(X)Z, (I)
CH₂═CH—OC(O)R; (II)
wherein:
X is chosen from a hydrogen atom and H and a methyl group;
Z is chosen from —C(O)OR¹, —C(O)NH₂, —C(O)NHR¹, —C(O)N(R¹)₂, —C₆H₅, —C₆H₄R¹, —C₆H₄OR¹, —C₆H₄Cl, —CN, —NHC(O)CH₃, —NHC(O)H, N-(2-pyrrolidonyl), N-caprolactamyl, —C(O)NHC(CH₃)₃, —C(O)NHCH₂CH₂—NH—CH₂CH₂-urea, —Si(R)₃, —C(O)O(CH₂)ₓSi(R)₃, —C(O)NH(CH₂)ₓSi(R)₃ and —(CH₂)ₓSi(R)₃;
x is an integer ranging from 1 to 6;
each R is independently chosen from a C₁-C₃₀ alkyl group;
each R¹ is independently chosen from a C₁-C₃₀ alkyl group, a hydroxylated C₂-C₃₀ alkyl group, and a halogenated C₁-C₃₀ alkyl groups

6. The composition according to claim 5, wherein the at least one hydrophobic non-ionic vinylic monomer is chosen from C₁-C₃₀ alkyl(meth)acrylates, (C₁-C₃₀ alkyl)(meth)acrylamides, styrene, substituted styrenes, vinyl esters, unsaturated nitriles and unsaturated silanes.

7. The composition according to claim 1, wherein the at least one hydrophobic non-ionic vinylic monomer is present in an amount ranging from 20% to 80% by weight relative to the total weight of the mixture of monomers.

8. The composition according to claim 1, wherein the at least one associative vinylic monomer is chosen from compounds of formula (III): 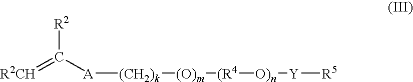
wherein:
each R² is independently chosen from a hydrogen atom, a methyl group, a —C(O)OH group, and a —C(O)OR³ group;
R³ is a C₁-C₃₀ alkyl group;
A is chosen from —CH₂C(O)O—, —C(O)O—, —O—, —CH₂O—, —NHC(O)NH—, —C(O)NH—, —Ar—(CE₂)_{z}-NHC(O)O—, —Ar—(CE₂)_{z}-NHC(O)NH— and —CH₂CH₂—NHC(O)—;
Ar is an arylene group;
E is chosen from a hydrogen atom and a methyl group;
z is an integer ranging from 0 to 1;
k is an integer ranging from 0 to 30;
m is an integer ranging from 0 to 1, with the provisos that when k is 0, then m is 0, and when k is an integer ranging from 1 to 30, then m is 1;
(R⁴—O)ₙ is a polyoxyalkylene group which is a homopolymer, a random copolymer or a block copolymer comprising C₂-C₄ oxyalkylene groups;
R⁴ is chosen from —C₂H₄—, —C₃H₆—, —C₄H₈— and mixtures thereof;
n is an integer ranging from 5 to 250;
Y is chosen from —R⁴O—, —R⁴NH—, —C(O)—, —C(O)NH—, R⁴NHC(O)NH—, and —C(O)NHC(O)—;
R⁵ is chosen from a substituted or unsubstituted alkyl group, chosen from linear C₈-C₄₀ alkyl groups, branched C₈-C₄₀ alkyl groups, C₈-C₄₀ alicyclic groups, phenyl groups substituted with a C₂-C₄₀ alkyl group, C₂-C₄₀ alkyl groups substituted with an aryl group, and C₈-C₈₀ complex esters;
wherein the alkyl group R⁵ optionally comprises at least one substituent chosen from hydroxy, alkoxy and halogeno groups.

9. The composition according to claim 8, wherein the at least one associative vinylic monomer is chosen from:
polyethoxylated cetyl(meth)acrylates,
polyethoxylated cetearyl(meth)acrylates,
polyethoxylated stearyl(meth)acrylates,
polyethoxylated arachidyl(meth)acrylates,
polyethoxylated behenyl(meth)acrylates,
polyethoxylated lauryl(meth)acrylates,
polyethoxylated cerotyl(meth)acrylates,
polyethoxylated montanyl(meth)acrylates,
polyethoxylated melissyl(meth)acrylates,
polyethoxylated lacceryl(meth)acrylates,
polyethoxylated 2,4,6-tri(1′-phenylethyl)phenyl(meth)acrylates,
polyethoxylated hydrogenated castor oil(meth)acrylates,
polyethoxylated canola(meth)acrylates,
polyethoxylated cholesterol(meth)acrylates,
and mixtures thereof,
wherein the polyethoxylated portion of the monomer comprises 5 to 100 ethylene oxide motifs.

10. The composition according to claim 1, wherein the at least one associative vinylic monomer is present in an amount ranging from 0.001% to 25% by weight relative to the total weight of the mixture of monomers.

11. The composition according to claim 1, wherein the mixture of monomers comprises at least one semi-hydrophobic vinylic surfactant monomer.

12. The composition according to claim 11, wherein the at least one semi-hydrophobic vinylic surfactant monomer is chosen from compounds of formulae (IV) and (V): 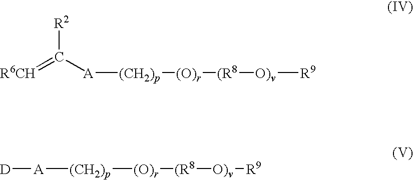
wherein:
each R⁶ is independently chosen from a hydrogen atom, a C₁-C₃₀ alkyl group, —C(O)OH, and —C(O)OR⁷;
R⁷ is a C₁-C₃₀ alkyl;
A is chosen from a —CH₂C(O)O—, —C(O)O—, —O—, —CH₂O—, —NHC(O)NH—, —C(O)NH—, —Ar—(CE₂)_{z}-NHC(O)O—, —Ar—(CE₂)_{z}-NHC(O)NH— and a —CH₂CH₂NHC(O)— group;
Ar is an arylene group;
E is chosen from a hydrogen atom and a methyl group;
z is an integer ranging from 0 to 1;
p is an integer ranging from 0 to 30;
r is an integer ranging from 0 to 1, with the provisos that when p is 0, then r is 0, and when p is an integer ranging from 1 to 30, then r is 1,
(R₈—O)ᵥ is a polyoxyalkylene which is a homopolymer, a random copolymer or a block copolymer with C₂-C₄ oxyalkylene motifs, wherein R⁸ is chosen from —C₂H₄—, —C₃H₆—, —C₄H₈— and mixtures thereof, and v is an integer ranging from 5 to 250;
R⁹ is chosen from a hydrogen atom and a C₁-C₄ alkyl group; and
D is chosen from C₈-C₃₀ alkenyl groups optionally substituted with a carboxy group.

13. The composition according to claim 11, wherein the at least one semi-hydrophobic vinylic surfactant monomer is present in an amount ranging from 0% to 25% by weight relative to the total weight of the mixture of monomers.

14. The composition according to claim 1, wherein the mixture of monomers comprises at least one hydroxylated non-ionic vinylic monomer.

15. The composition according to claim 1, wherein the mixture of polymers further comprises at least one hydroxylated non-ionic vinylic monomer chosen from C₁-C₆ hydroxyalkyl(meth)acrylates, (C₁-C₄ hydroxyalkyl)(meth)acrylamides and mixtures thereof.

16. The composition according to claim 14, wherein the hydroxylated non-ionic vinylic monomer is present in an amount ranging from 0% to 10% by weight relative to the total weight of the mixture of monomers.

17. The composition according to claim 1, wherein the mixture of monomers comprises at least one cross-linking monomers present in an amount ranging from 0.001% to 5% by weight relative to the total weight of the mixture of monomers.

18. The composition according to claim 1, wherein the mixture of monomers at least one chain transfer agent present in an amount ranging from 0% to 10% by weight relative to the total weight of the mixture of monomers.

19. The composition according to claim 1, wherein the at least one cationic polymer (i) is obtained by polymerizing a mixture of monomers comprising, with respect to the total weight of the mixture of monomers:
a) from 10% to 70% by weight of at least one vinylic monomer substituted with at least one amino group;
b) from 20% to 80% by weight of at least one hydrophobic non-ionic vinylic monomer;
c) from 0.001% to 25% by weight of at least one associative vinylic monomer;
d) from 0% to 25% by weight of at least one semi-hydrophobic vinylic surfactant monomer;
e) from 0% to 10% by weight of at least one hydroxylated non-ionic vinylic monomer;
f) from 0% to 5% by weight of at least one cross-linking monomer;
g) from 0% to 10% by weight of at least one chain transfer agent; and
h) from 0% to 2% by weight of at least one polymeric stabilizing agent.

20. The composition according to claim 1, wherein the at least one cationic polymer (i) is obtained by polymerizing a mixture of monomers comprising, with respect to the total weight of the mixture of monomer:
a) from 20% to 60% by weight of at least one vinylic monomer substituted with at least one amino group;
b) from 20% to 70% by weight of at least one hydrophobic non-ionic vinylic monomer;
c) from 0.01% to 15% by weight of at least one associative vinylic monomer;
d) from 0.1% to 10% by weight of at least one semi-hydrophobic vinylic surfactant monomer;
e) from 0.01% to 10% by weight of at least one hydroxylated non-ionic vinylic monomer;
f) from 0.001% to 5% by weight of at least one cross-linking monomer;
g) from 0.001% to 10% by weight of at least one chain transfer agent; and
h) from 0% to 2% by weight of at least one polymeric stabilizing agent.

21. The composition according to claim 1, wherein the cationic polymer (i) is obtained by polymerizing the following mixture of monomers:
a di(C₁-C₄ alkyl)amino(C₁-C₆ alkyl)methacrylate;
at least one ester of C₁-C₃₀ alkyl and (meth)acrylic acid;
a C₁₀-C₃₀ alkyl methacrylate polyethoxylated comprising from 20 to 30 moles of ethylene oxide;
an allyl ether of polyethylene glycol/polypropylene glycol, 30/5;
a hydroxy(C₂-C₆ alkyl)methacrylate; and
an ethylene glycol dimethacrylate.

22. The composition according to claim 1, wherein the cationic polymer (i) is a thickening polymer.

23. The composition according to claim 1, wherein the at least one cationic polymer (i) is present in an amount ranging from 0.01% to 10% by weight relative to the total composition weight.

24. The composition according to claim 1, wherein the at least one ester of the C₈ to C₂₄ fatty acid and oxyethylenated sorbitan is an ester of a C₁₂ fatty acid and oxyethylenated sorbitan comprising 2 to 10 oxyethylene units.

25. The composition according to claim 1, wherein the at least one ester of the C₈ to C₂₄ fatty acid and oxyethylenated sorbitan is 40E oxyethylenated sorbitan monolaurate.

26. The composition according to claim 1, wherein the at least one ester of a C₈-C₂₄ fatty acid and oxyethylenated sorbitan comprising 2 to 10 oxyethylene motifs is present in an amount ranging from 0.5% to 10% by weight relative to the total composition weight.

27. The composition according to claim 1, further comprising at least one cyclodextrin chosen from α-cyclodextrin, β-cyclodextrin and γ-cyclodextrin, which may or may not be chemically modified.

28. The composition according to claim 27, wherein said cyclodextrin is β-cyclodextrin.

29. The composition according to claim 27, wherein said cyclodextrin is present in amount ranging from 1% to 15% by weight relative to the total composition weight.

30. The composition according to claim 1, further comprising at least one surfactant chosen from anionic, non-ionic, and amphoteric surfactants, and mixtures thereof.

31. The composition according to claim 30, comprising at least one surfactant is chosen from at least one anionic surfactant and at least one amphoteric and zwitterionic surfactant.

32. The composition according to claim 30, wherein the anionic surfactant is chosen from alkylsulfates, alkylethersulfates, and mixtures thereof.

33. The composition according to claim 30, wherein the at least one anionic surfactant is present in an amount ranging from 0.5% to 50% by weight relative to the total composition weight.

34. The composition according to claim 31 wherein the at least one amphoteric and zwitterionic surfactant are chosen from (C₈-C₂₀ alkyl)betaines, (C₈-C₂₀ alkyl)amido(C₆-C₈ alkyl)betaines, and mixtures thereof.

35. The composition according to claim 1, further comprising at least one cationic surfactant.

36. The composition according to claim 1, further comprising at least one cationic polymer which is different from the at least one cationic polymer (i).

37. The composition according to claim 1, wherein the cosmetically acceptable medium is constituted by water or a mixture of water and at least one cosmetically acceptable solvent.

38. The composition according to claim 1, wherein the pH is less than 7.

39. A method for treating keratinous materials, comprising applying to the keratinous materials a composition comprising, in a cosmetically acceptable medium:
(i) at least one cationic polymer obtained by polymerizing a mixture of monomers comprising at least one vinylic monomer substituted with at least one amino group, at least one hydrophobic non-ionic vinylic monomer, and at least one associative vinylic monomer, and;
(ii) at least one ester of a C₈-C₂₄ fatty acid and oxyethylenated sorbitan comprising 2 to 10 oxyethylene motifs,
optionally followed by rinsing with water, after an optional leave-in time.

40. The composition according to claim 1, wherein the composition is in the form of a shampoo.

41. The composition according to claim 1, wherein the composition is in the form of a conditioner.
